(19) United States
(12) Patent Application Publication
(10) Pub. No.: US 2026/0367096 A1
(43) Pub. Date: Nov. 19, 2026

(54) BIOMARKER FOR IDIOPATHIC PULMONARY FIBROSIS AND APPLICATIONS THEREOF

(71) Applicant: Zhejiang Chinese Medical University, Zhejiang (CN)

(72) Inventors: Jiayi Xu, Zhejiang (CN); Huifen Zhou, Zhejiang (CN); Haitong Wan, Zhejiang (CN); Jiehong Yang, Zhejiang (CN); Yiwen Luo, Zhejiang (CN); Zixuan Li, Zhejiang (CN); Yi Zhou, Zhejiang (CN); Xiao He, Zhejiang (CN); Sixian Chen, Zhejiang (CN)

(73) Assignee: Zhejiang Chinese Medical University, Zhejiang (CN)

(21) Appl. No.: 19/212,595

(22) Filed: May 19, 2025

(51) Int. Cl. G01N 33/68; C12Q 1/34; G01N 33/50

(52) U.S. Cl. CPC G01N 33/6893; C12Q 1/34; G01N 33/5023; G01N 2333/4704; G01N 2333/4706; G01N 2333/98; G01N 2500/10; G01N 2800/12

(57) ABSTRACT

The present invention relates to the field of biotechnology, and discloses biomarkers for idiopathic pulmonary fibrosis (IPF) and applications thereof. The biomarkers comprise ADIRF, PTGFRN, and UPB1. These biomarkers were identified for the first time as alternative splicing (AS)-related biomarkers in IPF through comprehensive bioinformatics analyses. Three key genes associated with IPF were identified, and their potential molecular mechanisms involved in the pathogenesis of IPF were further elucidated. Moreover, the relevance of PTGFRN and UPB1 to pulmonary fibrosis was validated using a bleomycin-induced mouse model of pulmonary fibrosis. The identified biomarkers provide novel targets and potential strategies for the diagnosis and treatment of IPF.

### BACKGROUND

### Technical Field

The present invention pertains to the field of biotechnology, and more particularly to a biomarker associated with idiopathic pulmonary fibrosis (IPF) and its use in diagnosis, prevention, and treatment.

### Description of Related Art

Idiopathic pulmonary fibrosis (IPF) is a chronic lung disease characterized by the formation and proliferation of scar tissue within the lungs. The primary symptoms of IPF include fatigue, exertional dyspnea, and persistent cough, ultimately progressing to respiratory failure. IPF predominantly affects middle-aged and elderly individuals, typically presenting insidiously and often being diagnosed at advanced stages, with a median survival time of only 3.8 years.

Currently, nintedanib and pirfenidone are the principal drugs recommended by clinical practice guidelines for the treatment of IPF. However, pharmacological treatment carries significant risks, including gastrointestinal disorders, cardiovascular events, and hepatic injury. The pathogenesis of IPF is highly complex and involves multiple contributing factors such as inflammation, epithelial-mesenchymal transition, and oxidative stress. Nevertheless, the precise pathogenic and molecular mechanisms underlying IPF remain incompletely understood. Accordingly, elucidating the exact mechanisms driving the onset and progression of IPF and developing more effective therapeutic strategies remain critical areas of current research.

Alternative splicing (AS) is a fundamental mechanism in eukaryotic RNA processing through which different combinations of exons are joined to generate multiple distinct mRNA transcripts from a single gene, thereby significantly increasing the complexity of the proteome. It is estimated that more than 90% of human expressed genes undergo AS events. Dysregulation of AS plays a critical role in the development of various cancers and diseases by influencing key biological processes such as cell proliferation, apoptosis, invasion, migration, and metabolism. Studies have shown that a hallmark pathological feature of idiopathic pulmonary fibrosis (IPF) is the presence of fibroblastic foci, composed of clusters of myofibroblasts and the connective tissue matrix they deposit. SF2/ASF has been found to regulate the alternative splicing of fibronectin, a process shown to be essential for myofibroblast differentiation. Moreover, AS of the RAGE gene leads to the production of endogenous secretory RAGE (esRAGE), which acts as a decoy receptor to neutralize RAGE ligands. Notably, expression of esRAGE is decreased in the lung tissues of patients with IPF, suggesting that AS plays a pivotal role in the pathophysiology of IPF. However, the precise role of AS in IPF and its regulatory mechanisms remain poorly understood.

Mendelian randomization (MR) is an analytical approach that leverages genetic variants as instrumental variables to robustly infer causal relationships between exposures and outcomes. The primary advantage of MR lies in its reliance on the random allocation of genetic variants according to Mendel's laws of inheritance. By examining the distribution of alleles across a population, MR enables causal inference between genotypes and phenotypes, thereby overcoming key limitations of traditional observational studies. In recent years, MR has been increasingly applied in gene expression studies to identify the causal effects of specific genes on various traits or diseases, thereby enhancing our understanding of gene function and regulatory mechanisms.

In summary, the lack of novel targets, innovative approaches, and effective therapeutic agents for the prevention and treatment of idiopathic pulmonary fibrosis remains one of the most pressing challenges in the field of pulmonary fibrosis management.

### SUMMARY

In view of the deficiencies in the prior art, the present invention aims to provide a technical solution relating to a biomarker for idiopathic pulmonary fibrosis (IPF) and its applications.

The present invention is implemented through the following technical solutions:

In one aspect, the present invention provides a biomarker for idiopathic pulmonary fibrosis (IPF), wherein the biomarker comprises ADIRF, PTGFRN, and UPB1.

In another aspect, the present invention provides the use of the aforementioned biomarker and/or a detection reagent thereof in the preparation of a product for diagnosing idiopathic pulmonary fibrosis.

In a further aspect, the present invention provides a diagnostic kit for idiopathic pulmonary fibrosis, comprising reagents configured to detect the expression levels of the aforementioned biomarker associated with IPF.

In yet another aspect, the present invention provides the use of the aforementioned biomarker and/or a detection reagent thereof in screening candidate drugs or therapeutic methods for the prevention and/or treatment of idiopathic pulmonary fibrosis.

In a preferred embodiment, the screening comprises detecting the effect of a candidate drug or method on the expression levels of ADIRF, PTGFRN, or UPB1 in a subject, and selecting candidate drugs or methods capable of modulating the expression of one or more of these biomarkers for further validation.

In still another aspect, the present invention provides the use of the aforementioned biomarker and/or a regulator thereof in the preparation of a pharmaceutical composition for treating idiopathic pulmonary fibrosis.

Furthermore, in the aforementioned application, the regulatory agents comprise at least one selected from the group consisting of aflatoxin B1, benzo(a)pyrene, cyclosporin, retinoic acid, and valproic acid.

According to a sixth aspect of the present invention, a pharmaceutical composition is provided, wherein the composition comprises a regulatory agent of the idiopathic pulmonary fibrosis (IPF)-associated biomarkers described above.

In a further embodiment, the pharmaceutical composition additionally comprises a pharmaceutically acceptable excipient.

Compared with the prior art, the present invention offers at least the following advantageous effects:

The present invention, for the first time, identifies alternative splicing (AS)-related biomarkers in IPF using bioinformatics approaches. Three key genes associated with IPF (ADIRF, PTGFRN, and UPB1) were identified, and their molecular mechanisms contributing to IPF pathogenesis were further investigated.

The correlation between PTGFRN and UPB1 and pulmonary fibrosis was experimentally validated in a murine model of lung fibrosis, thereby providing novel therapeutic targets and directions for the treatment of IPF.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A shows the differences in SFRG scores between the IPF group and the control group.

FIG. 1B shows hierarchical clustering of samples after incorporating sample traits.

FIG. 1C shows the screening of soft thresholding powers.

FIGS. 1D1-1D2 shows a heatmap of the correlation between modules and phenotypes.

FIG. 2A is a volcano plot of differentially expressed genes between IPF and control groups.

FIG. 2B is a heatmap of differentially expressed genes between IPF and control groups.

FIG. 2C is a Venn diagram of candidate genes.

FIG. 2D shows a circular plot of GO enrichment results for candidate genes.

FIG. 2E shows a chord diagram of KEGG pathway enrichment for candidate genes.

FIGS. 3A-3K are leave-one-out sensitivity test forest plots of the causal relationship between each gene and IPF, including:

FIGS. 3A1-3A2: UPB1-IPF;

FIGS. 3B1-3B4: CA2-IPF;

FIG. 3C: PTGFRN-IPF;

FIG. 3D: IRF4-IPF;

FIG. 3E: GPR17-IPF;

FIG. 3F: ADIRF-IPF;

FIG. 3G: MPP3-IPF;

FIG. 3H: BATF2-IPF;

FIGS. 311-313: FAXDC2-IPF;

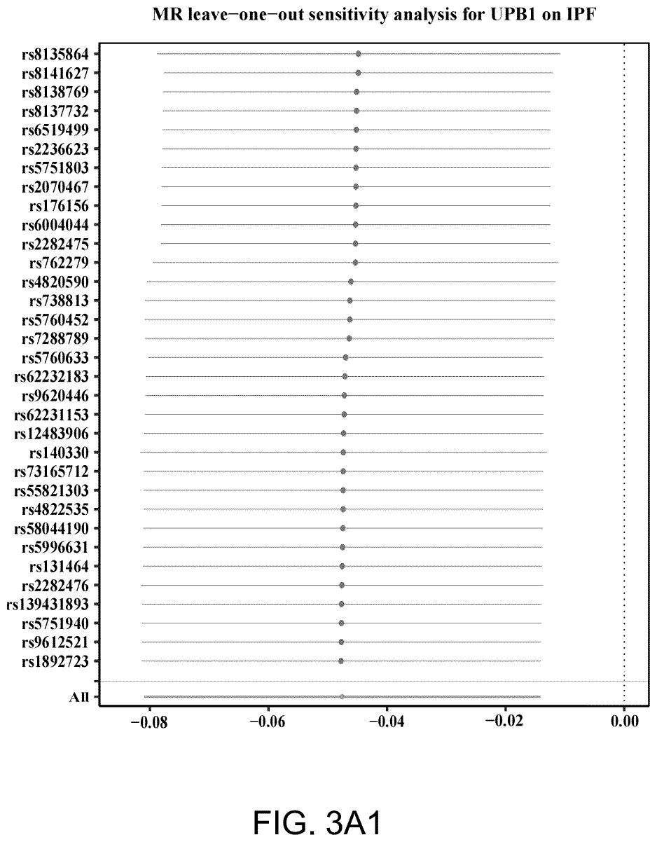

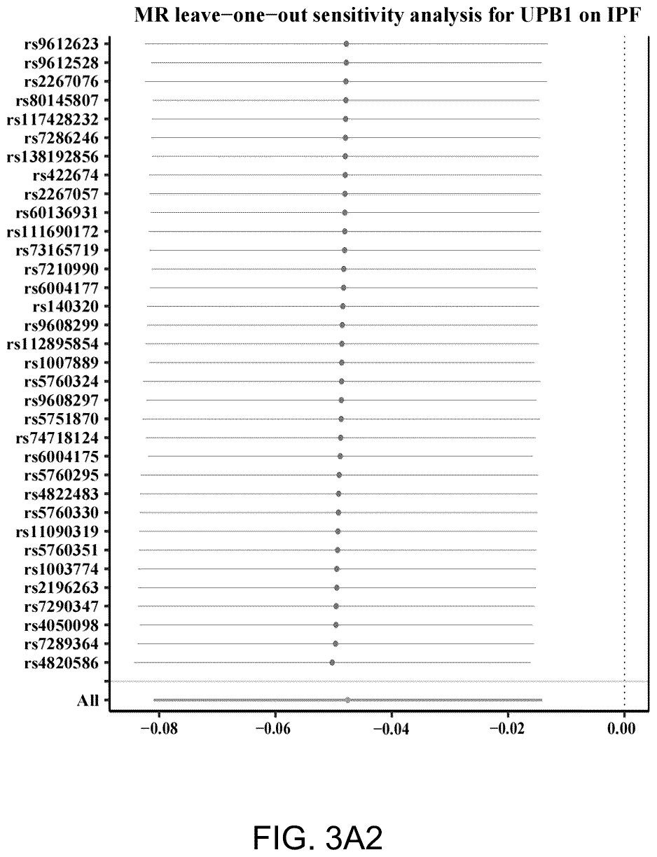

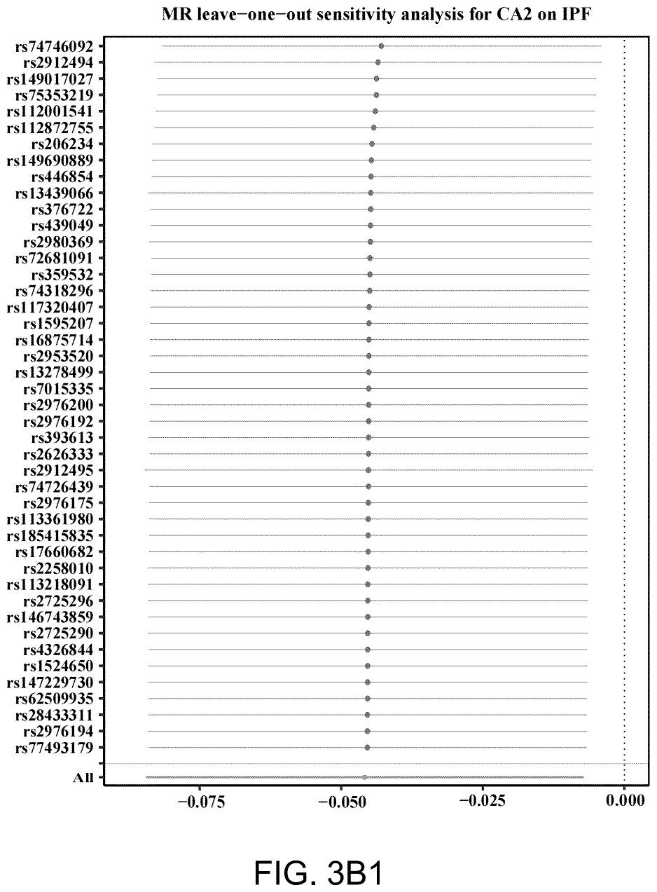

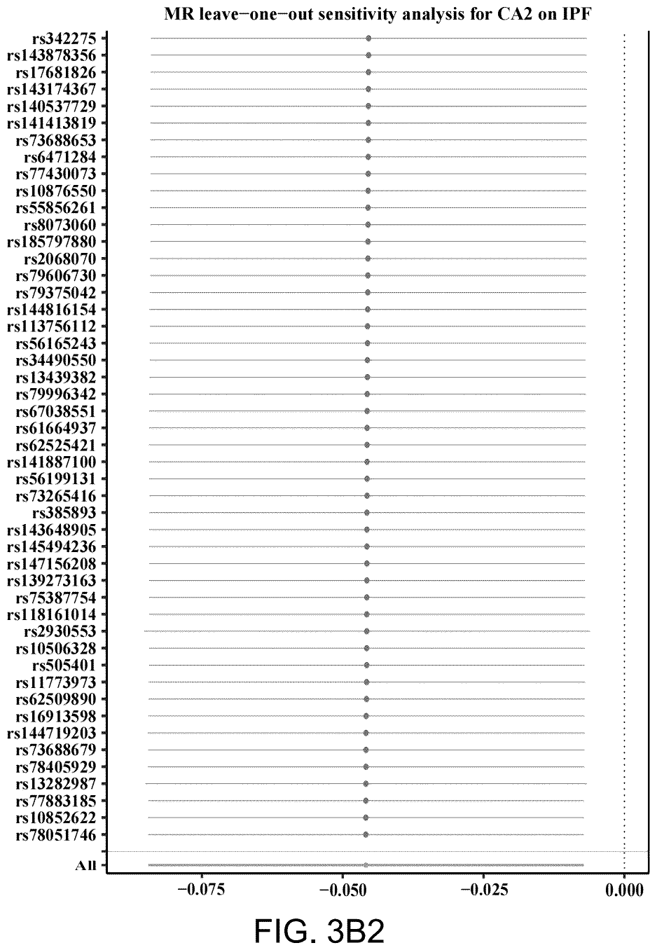

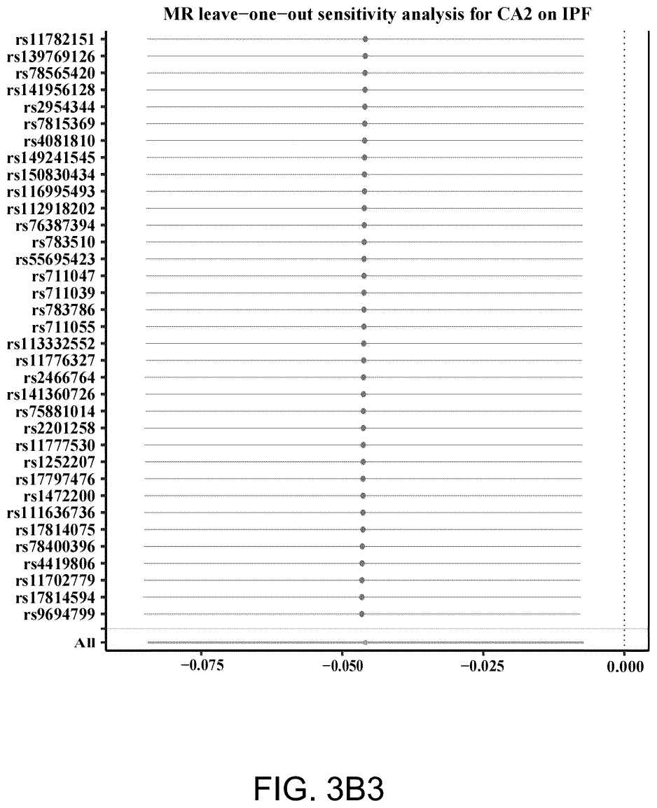

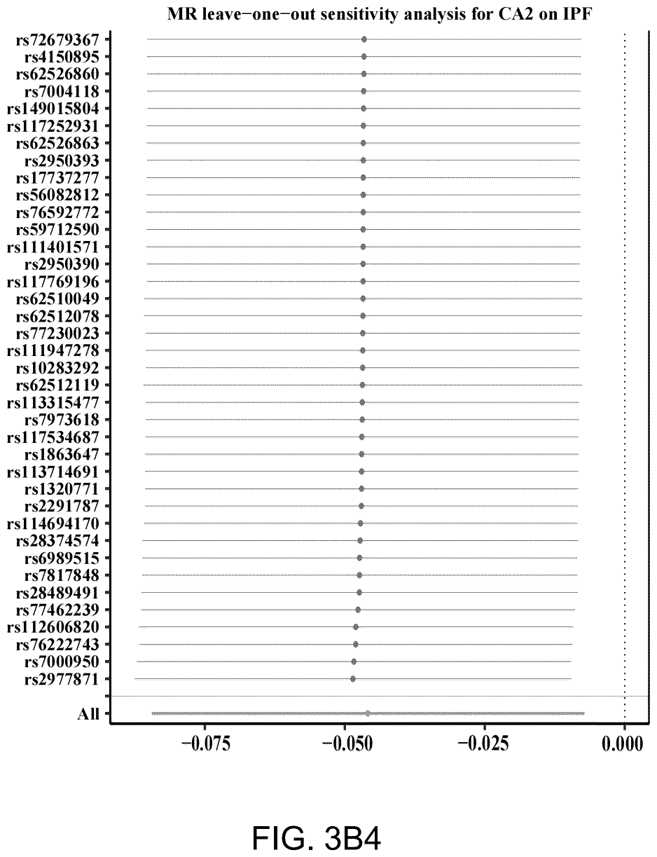

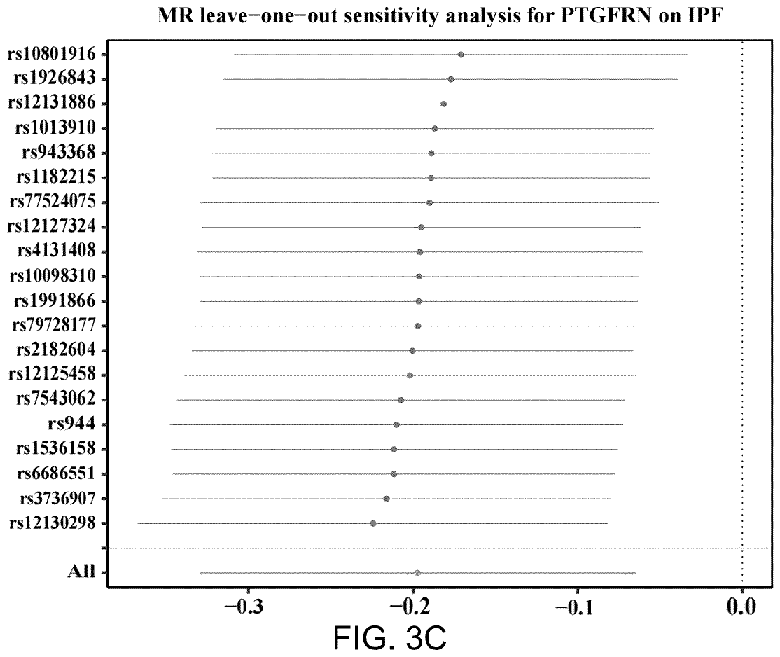

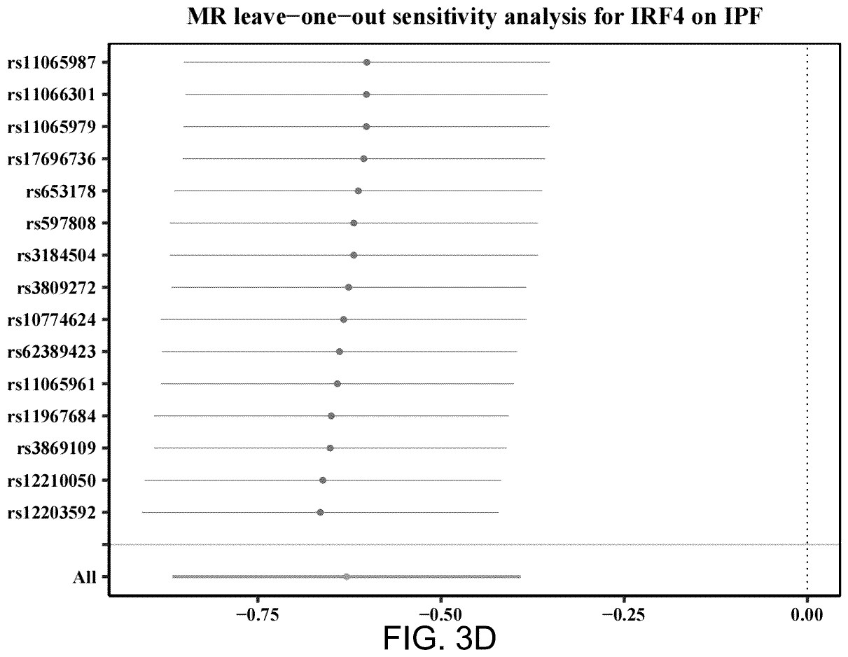

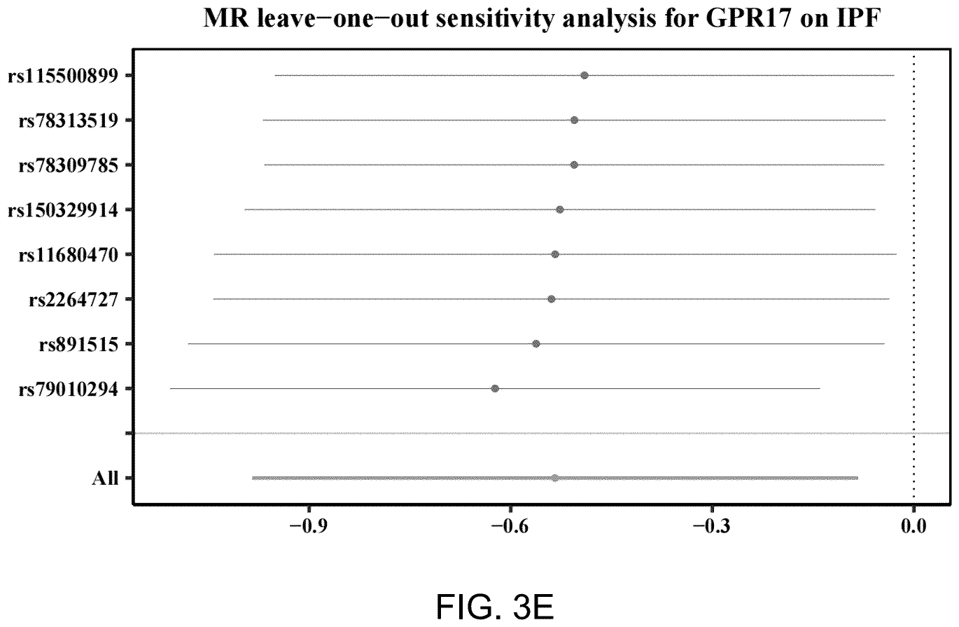

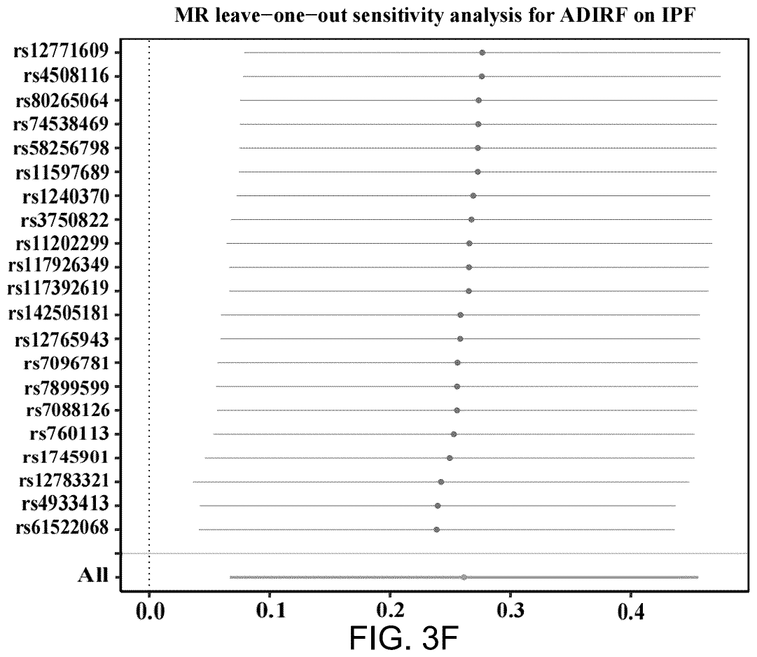

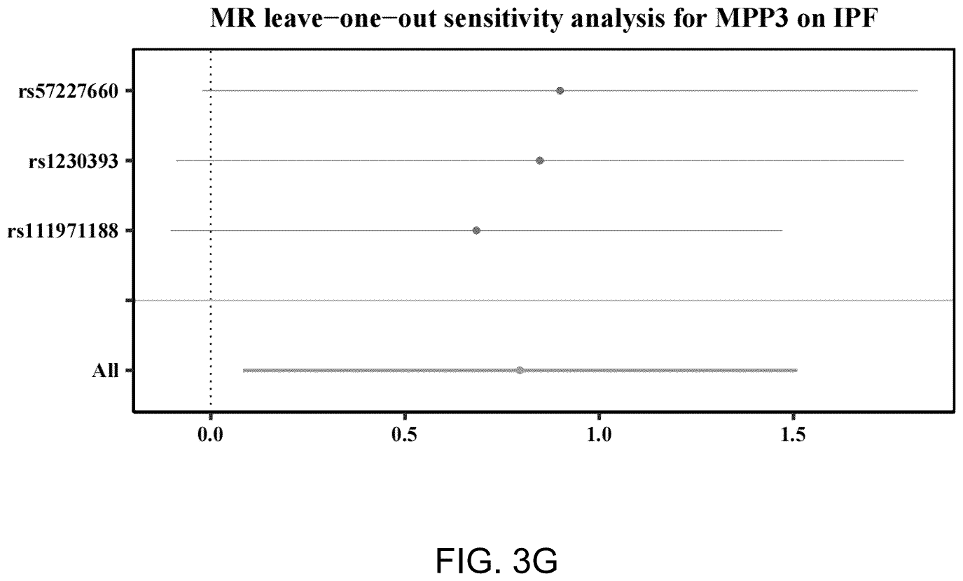

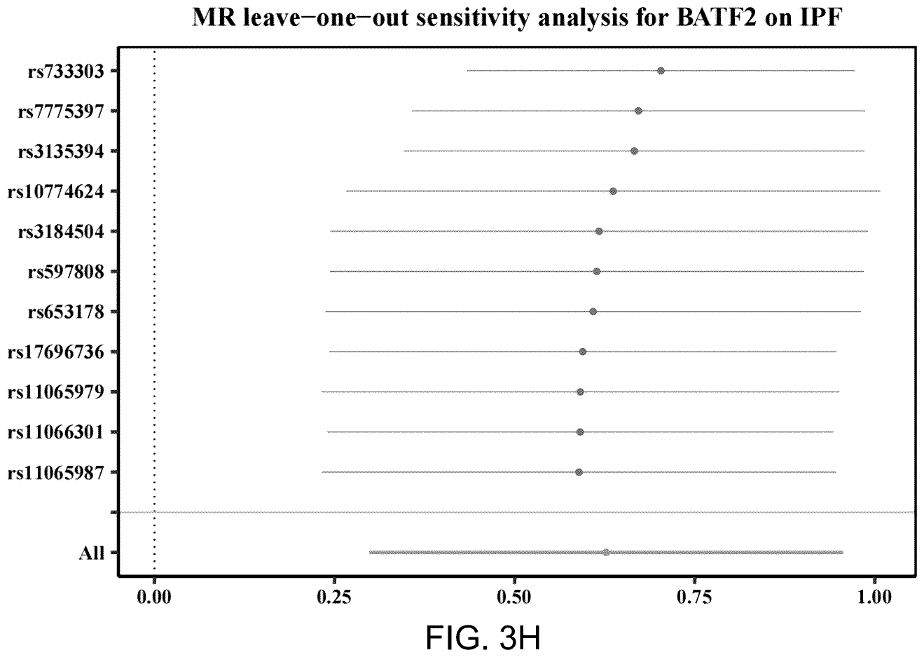

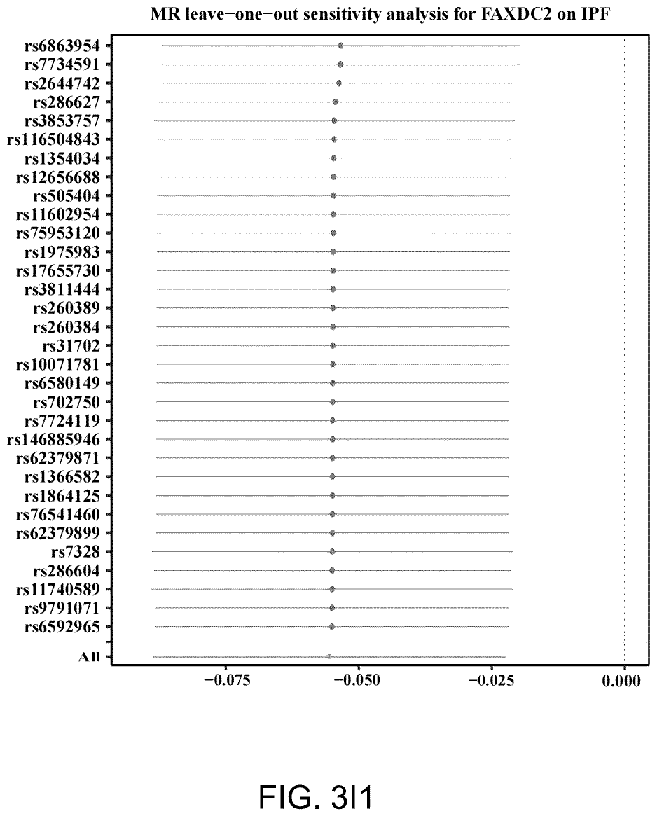

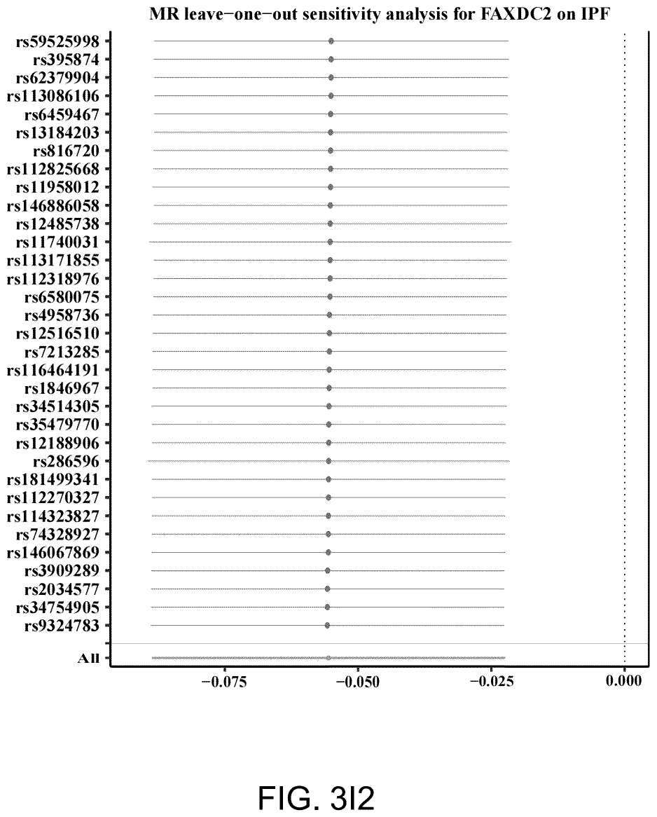

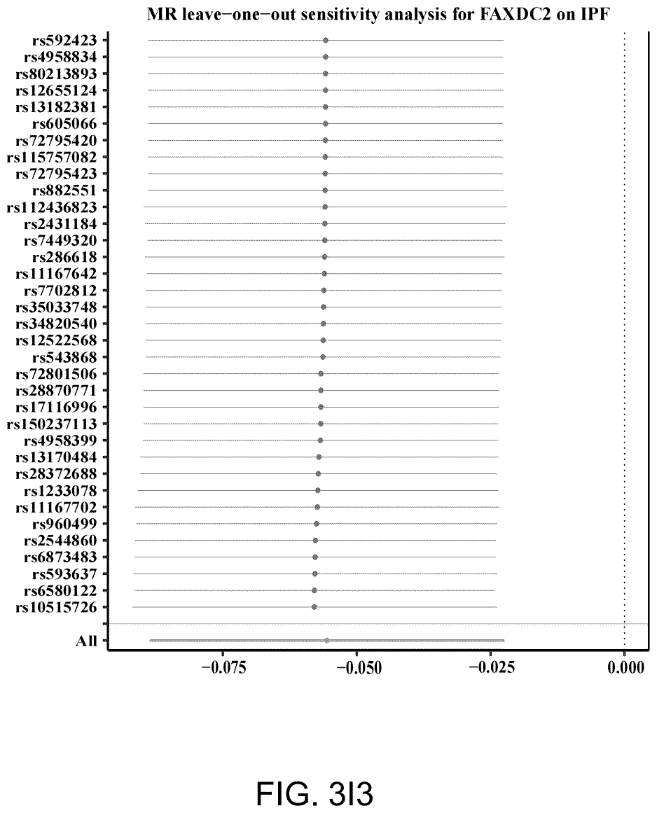

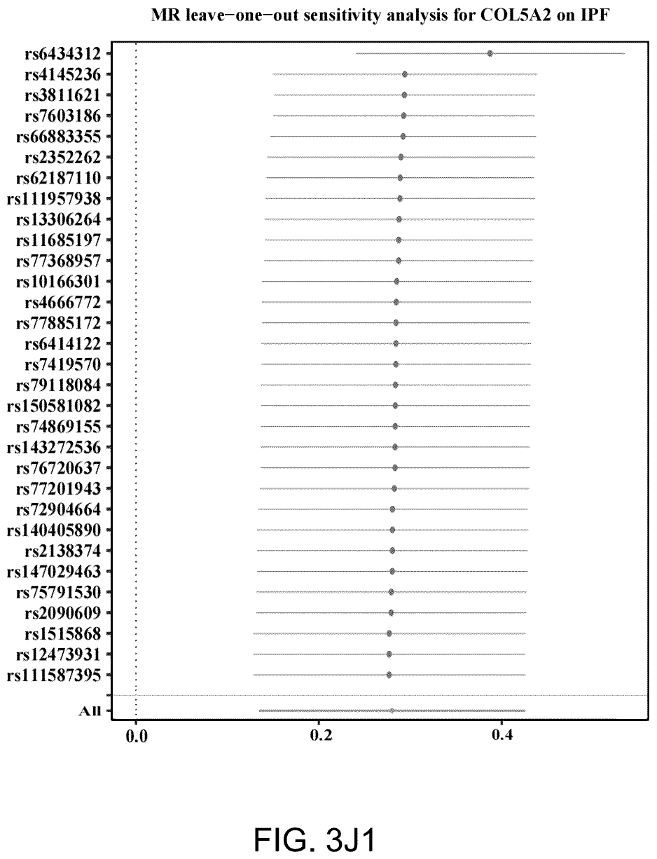

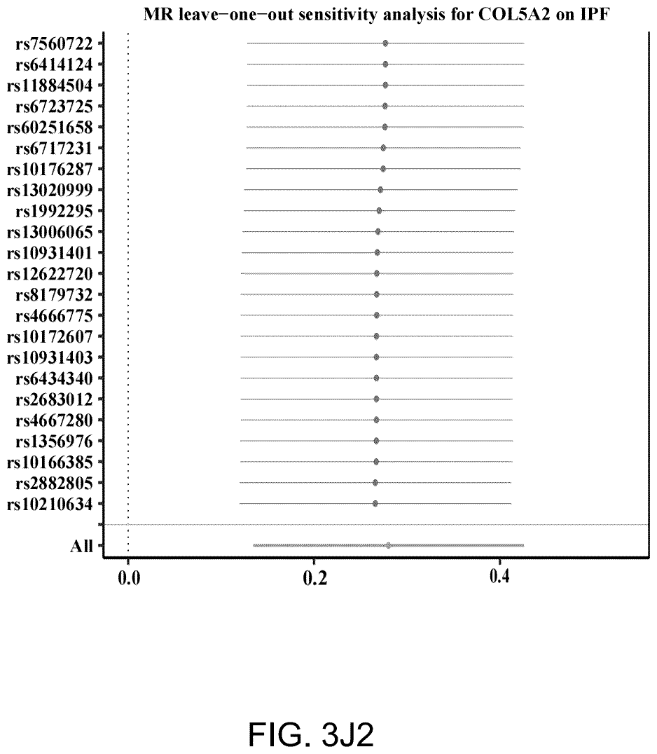

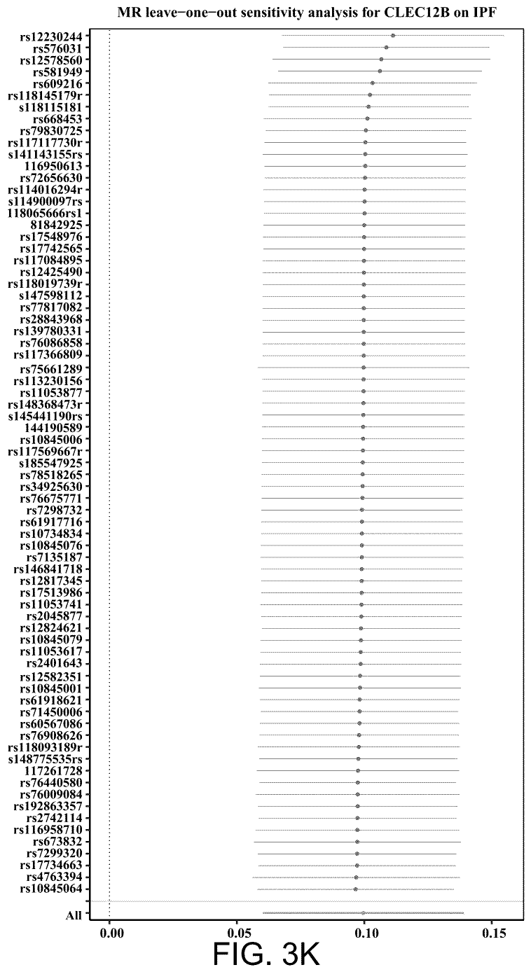

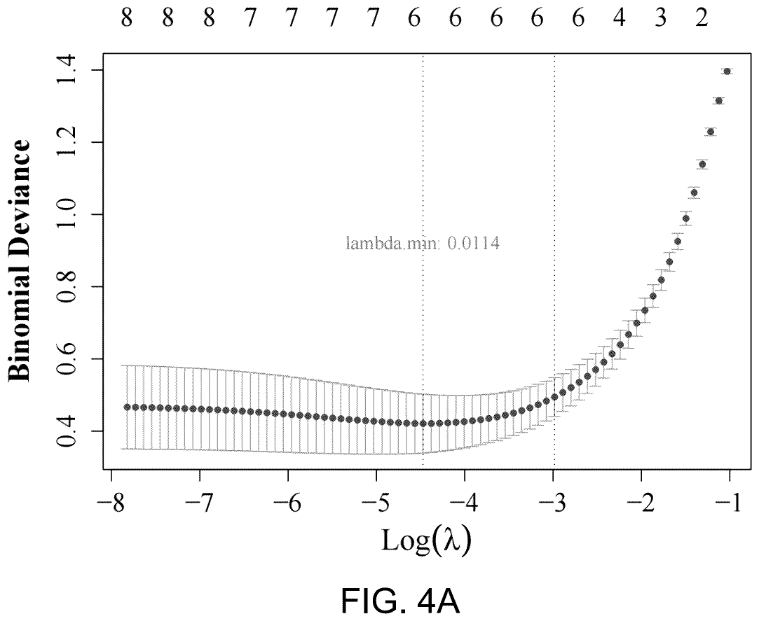

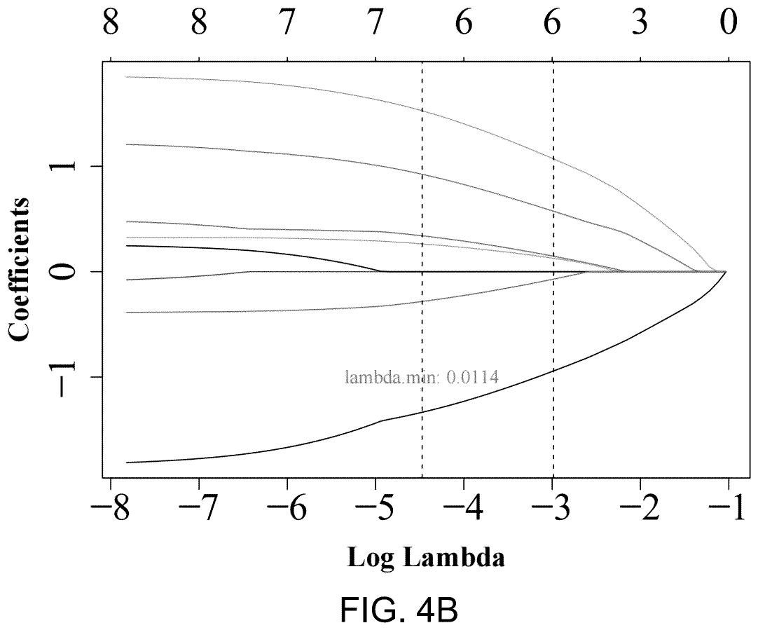

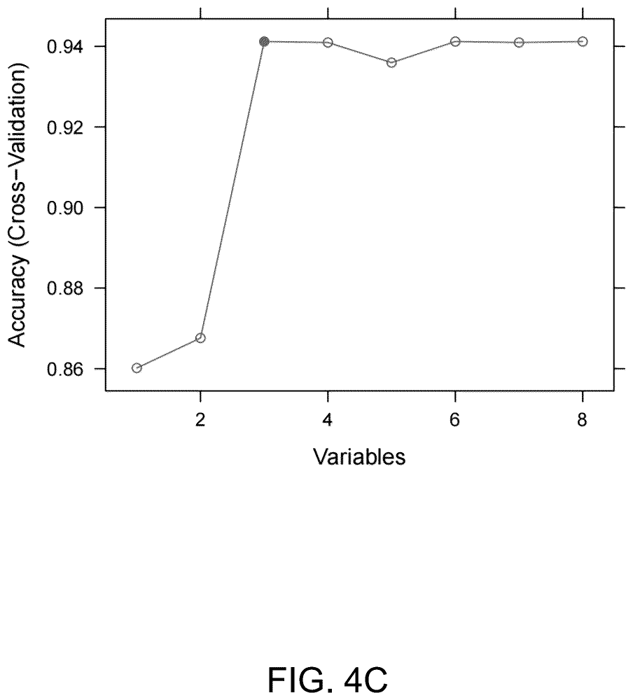

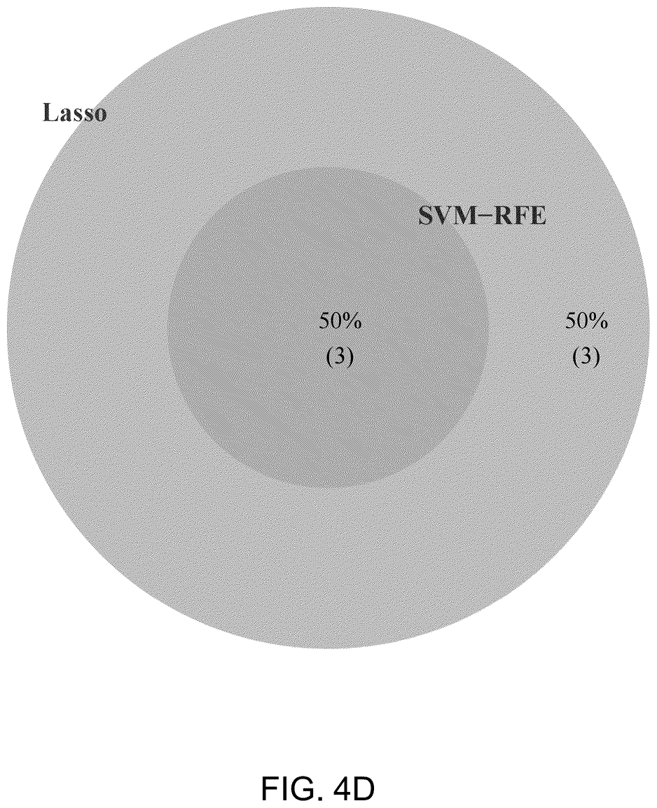

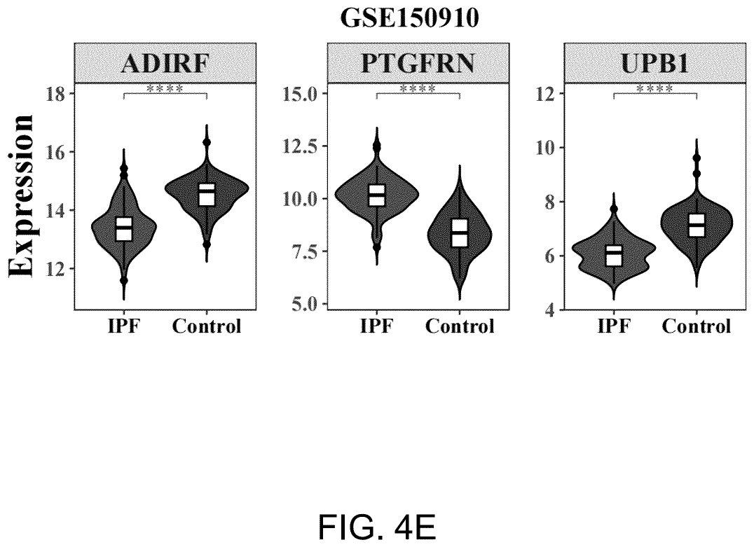

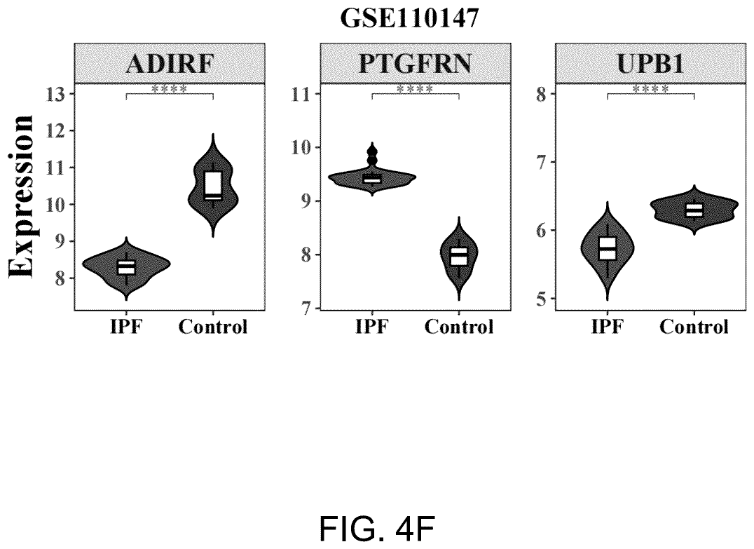

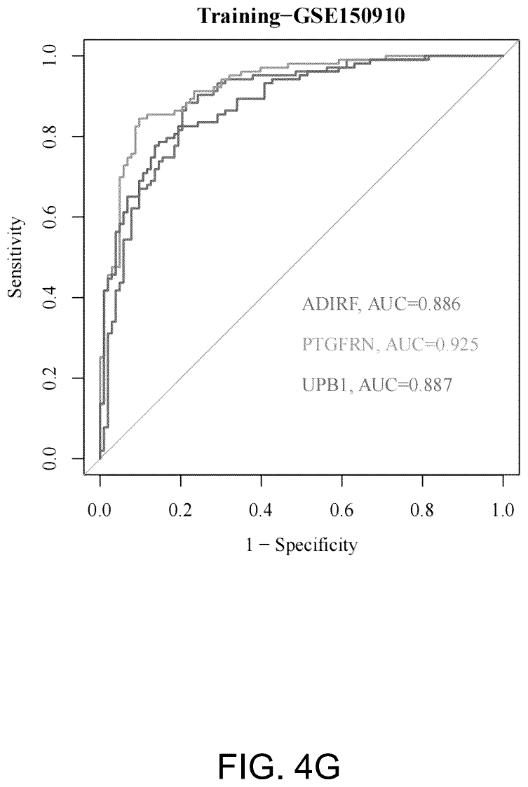

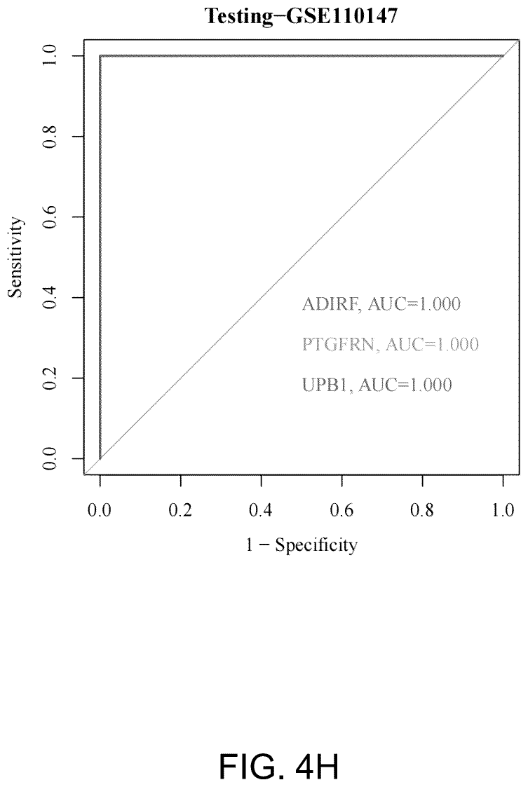

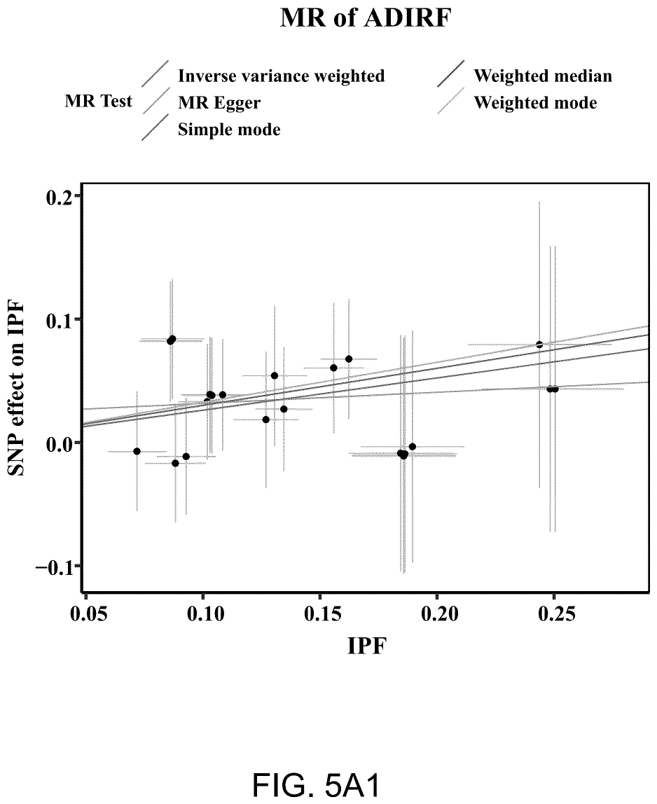

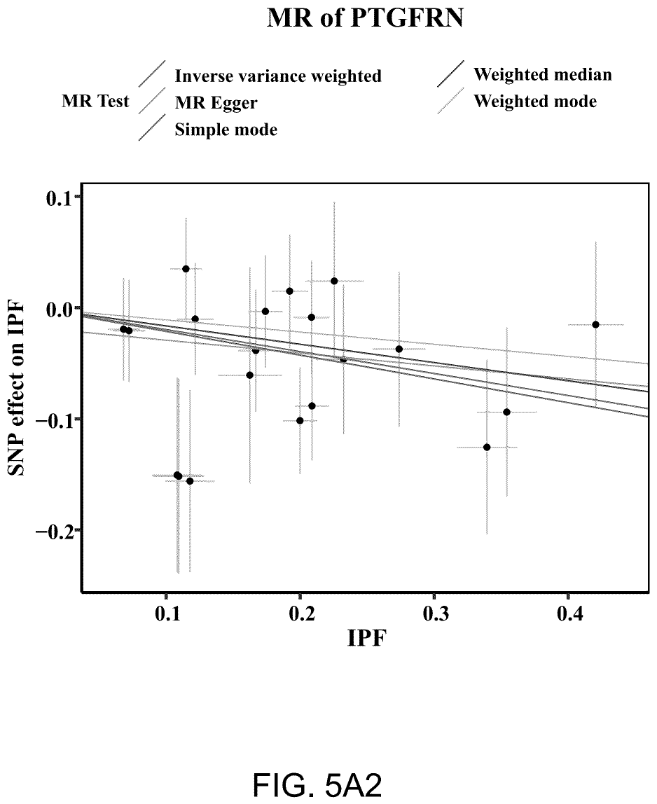

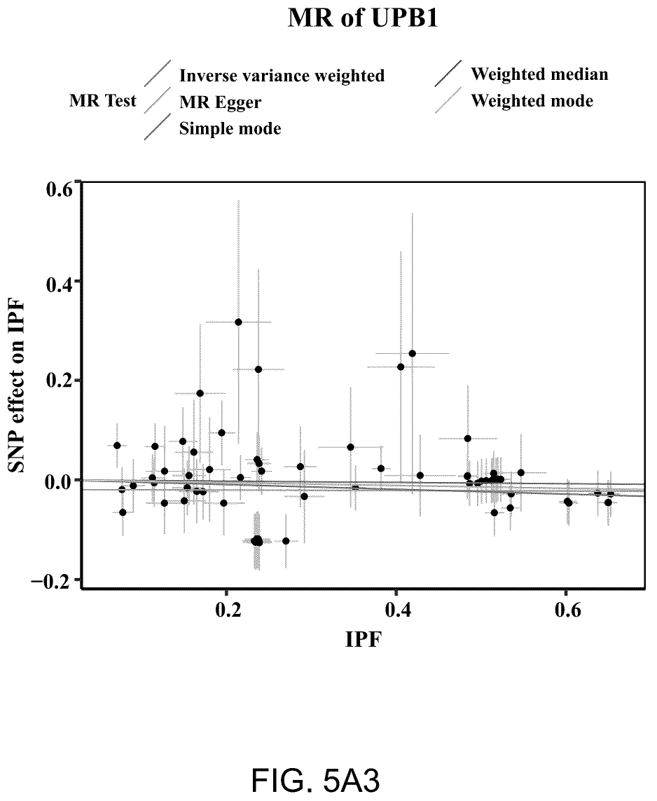

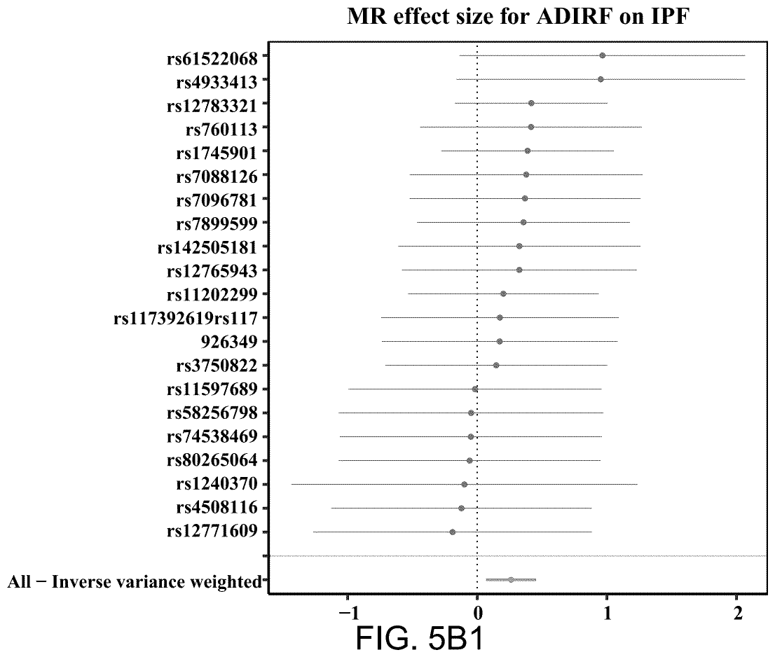

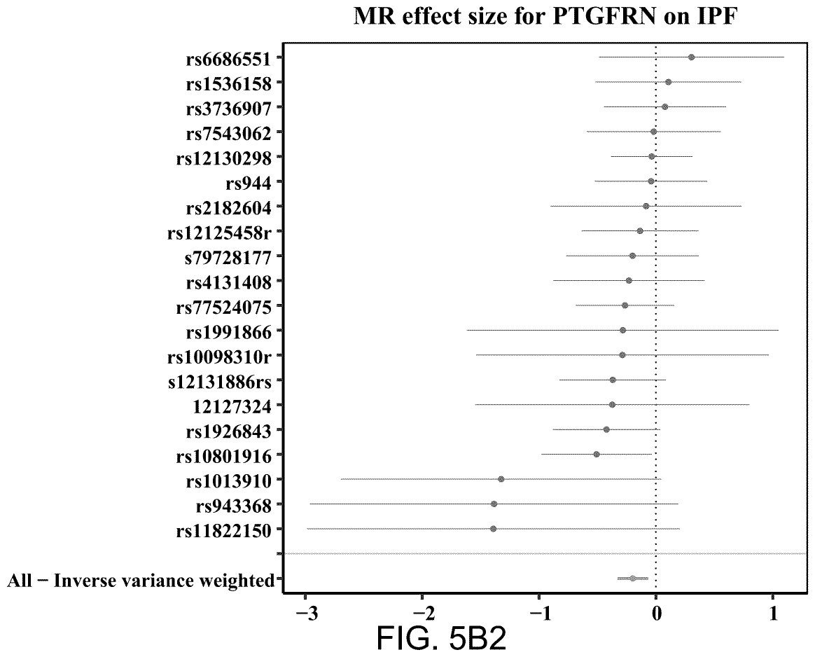

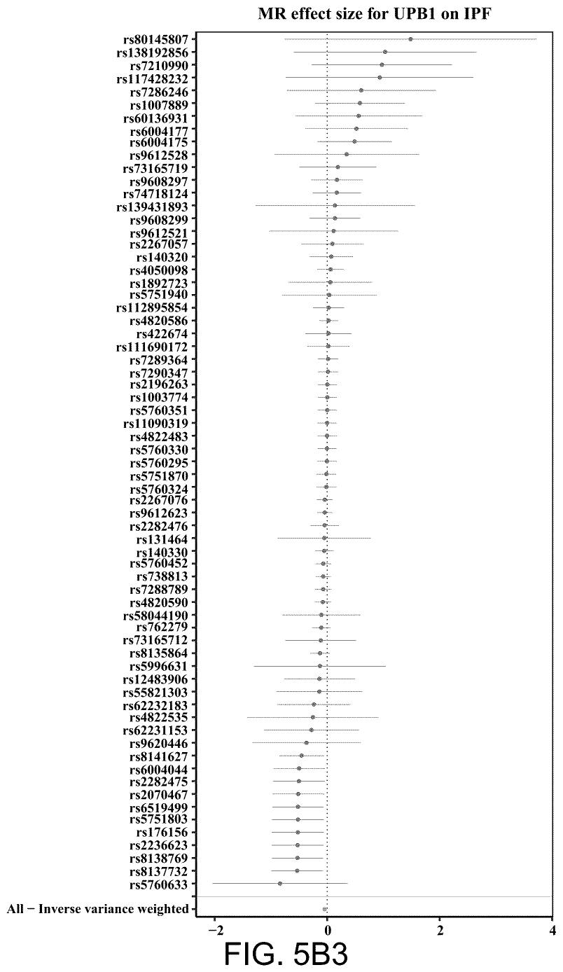

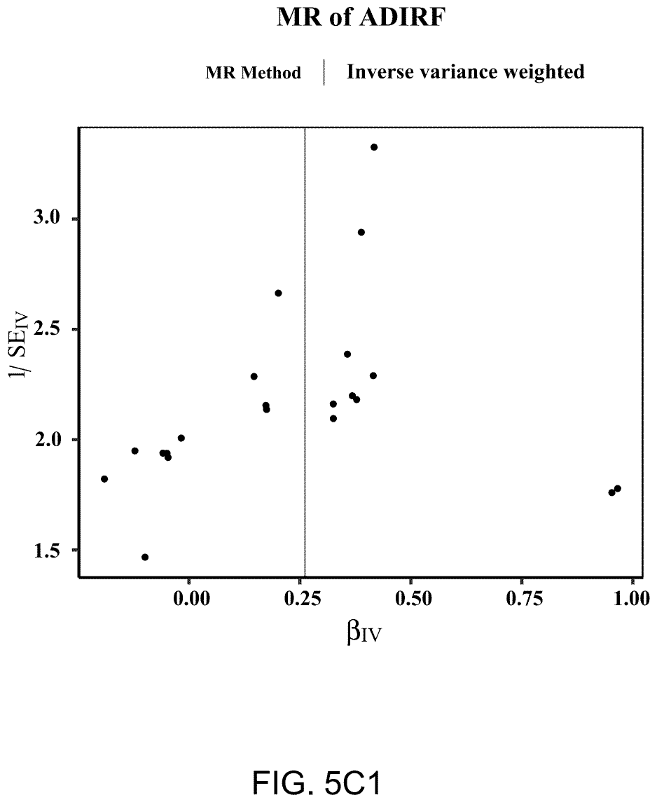

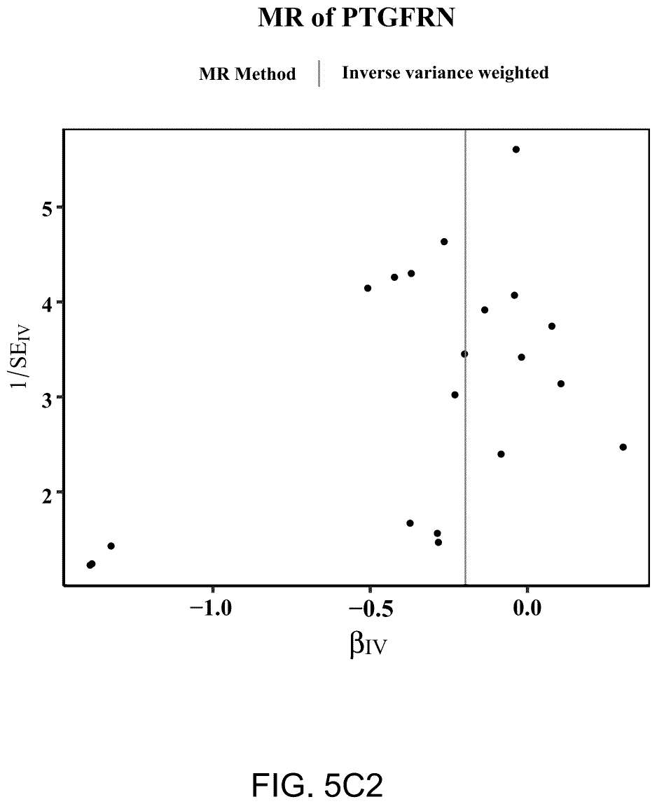

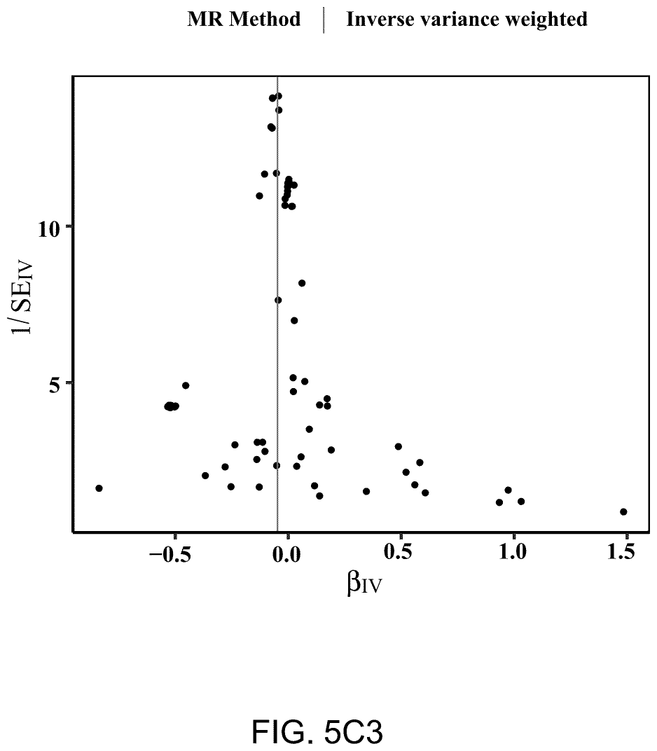

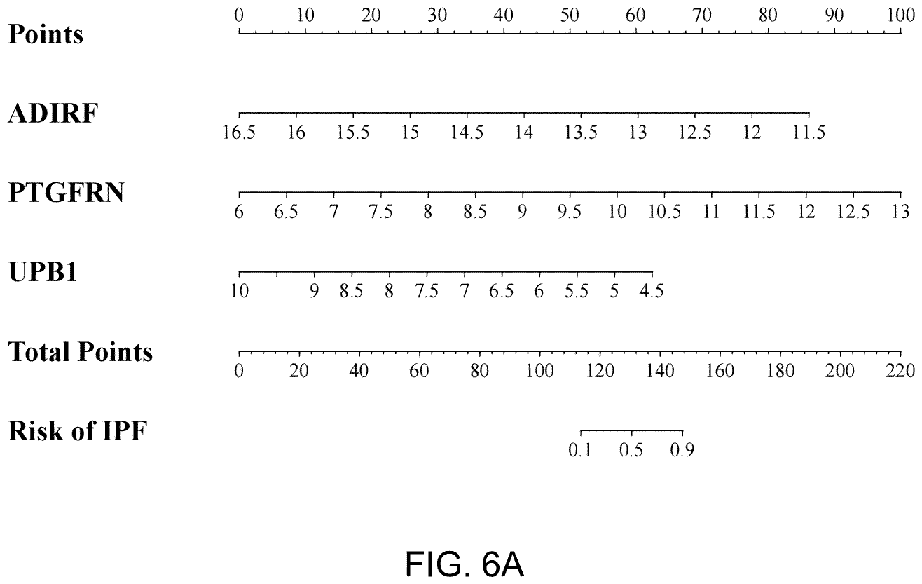

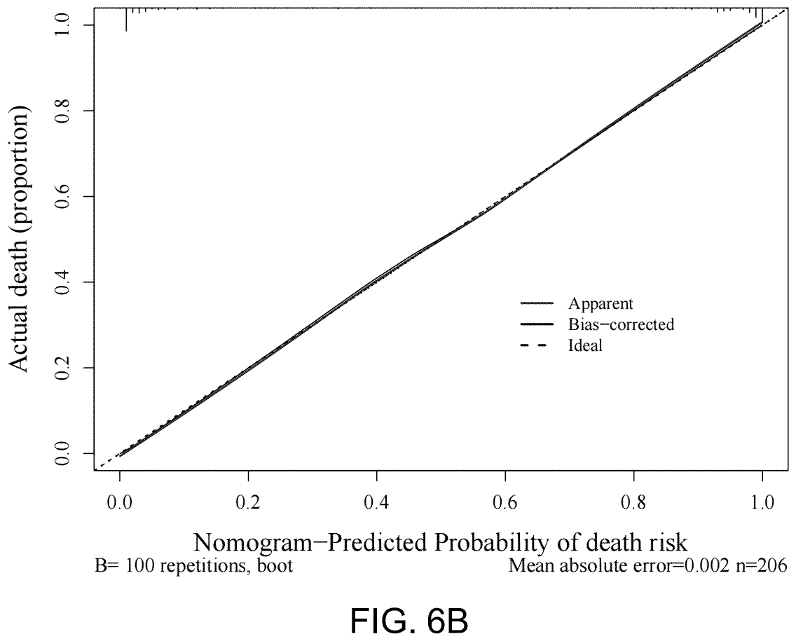

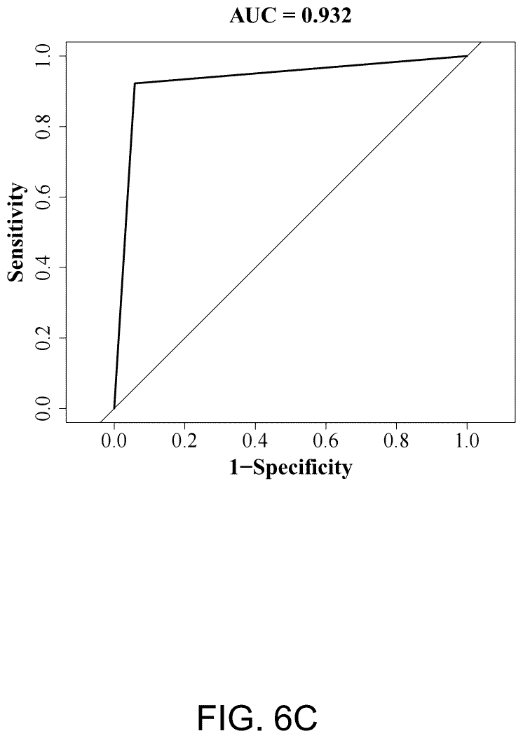

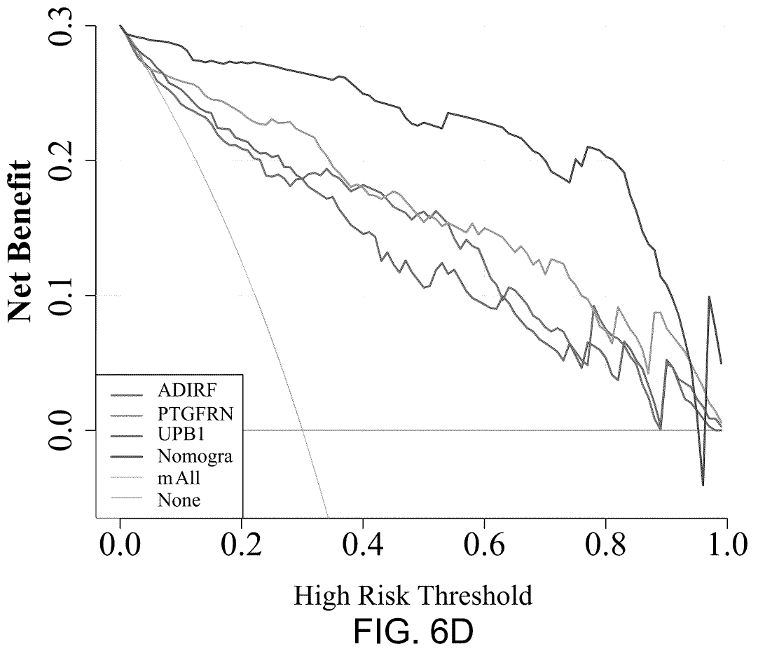

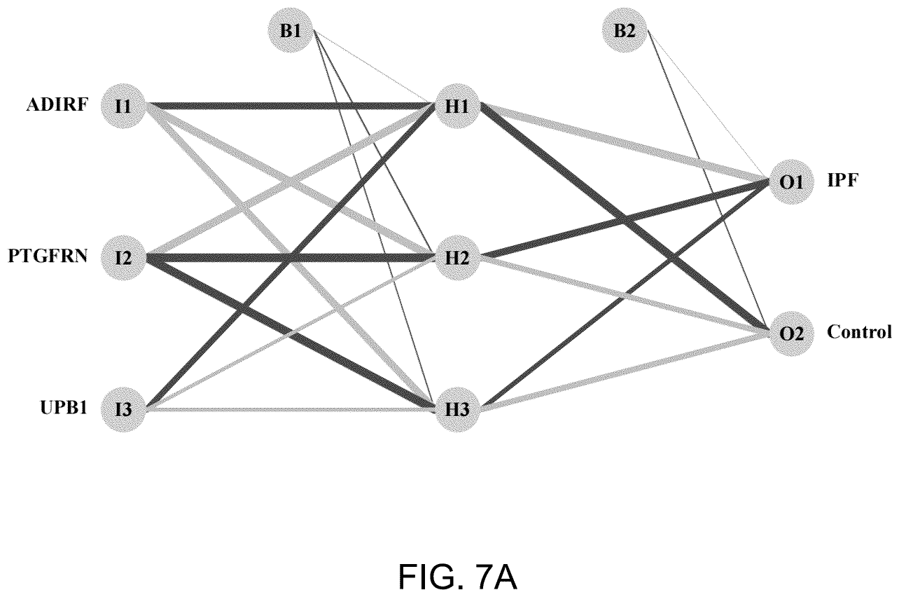

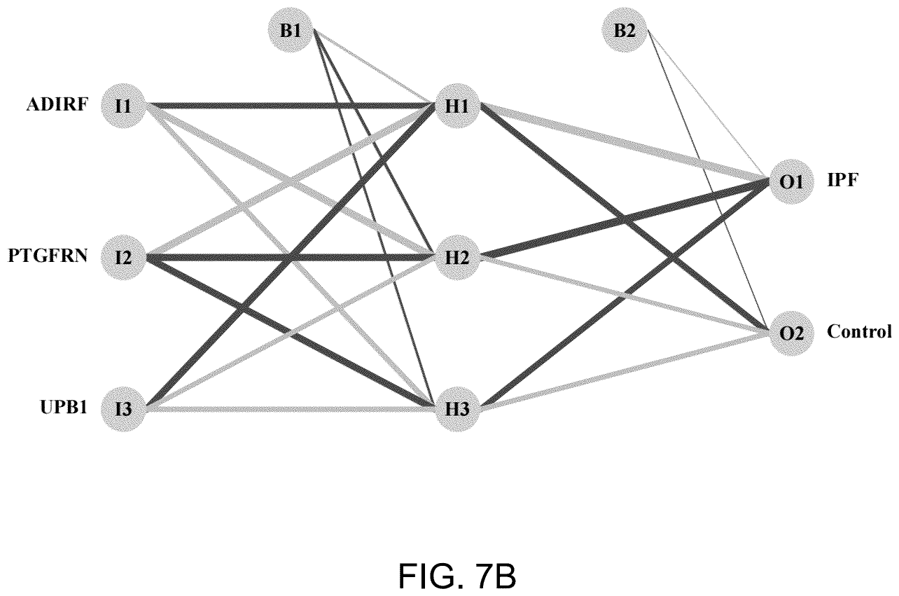

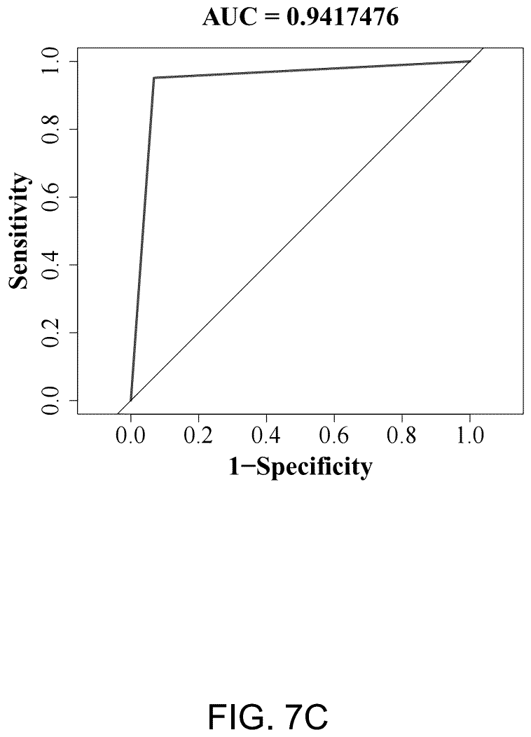

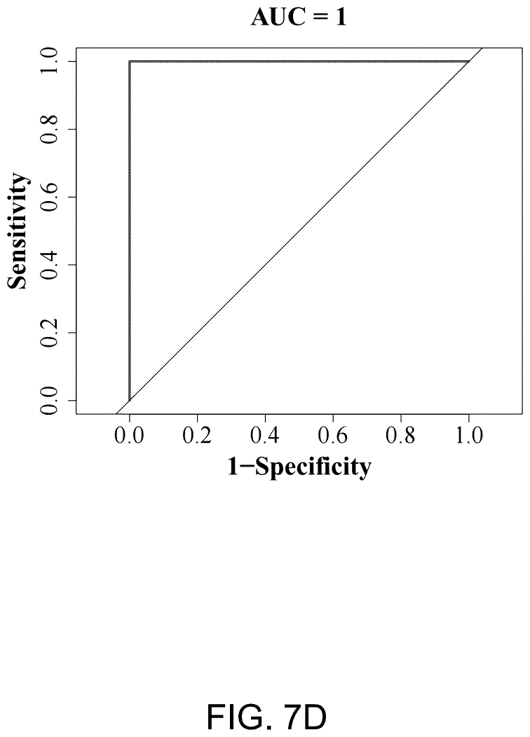

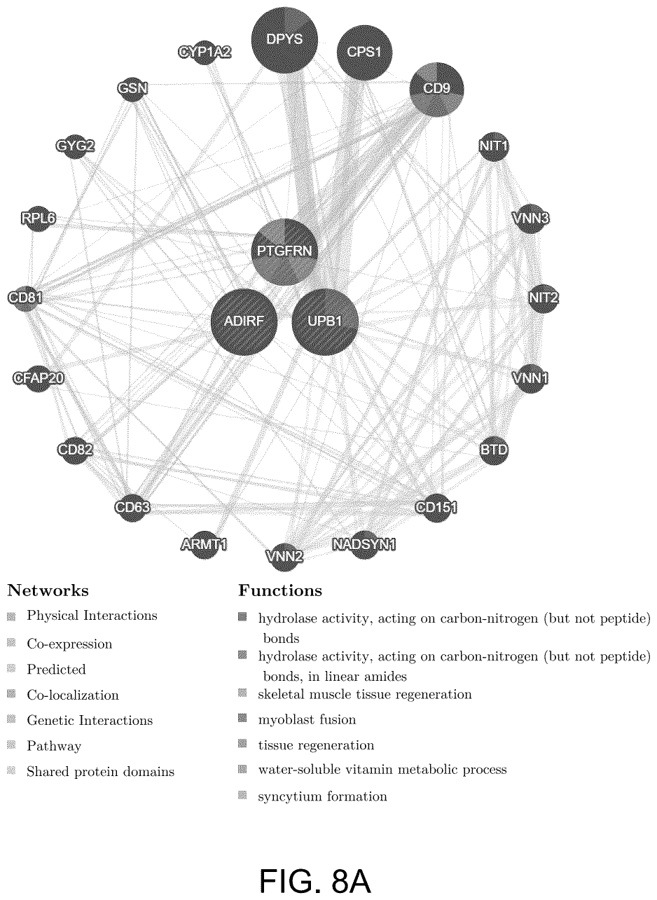

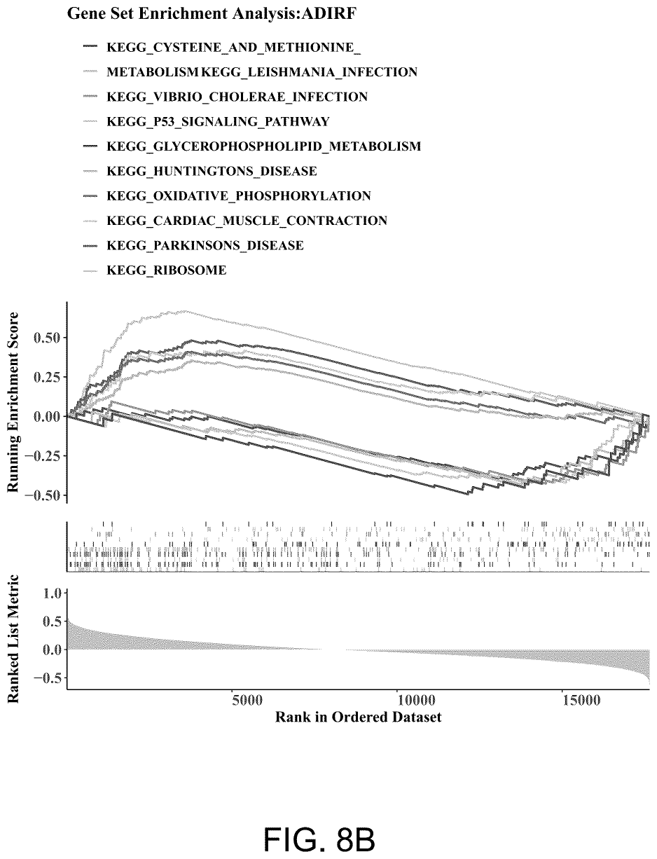

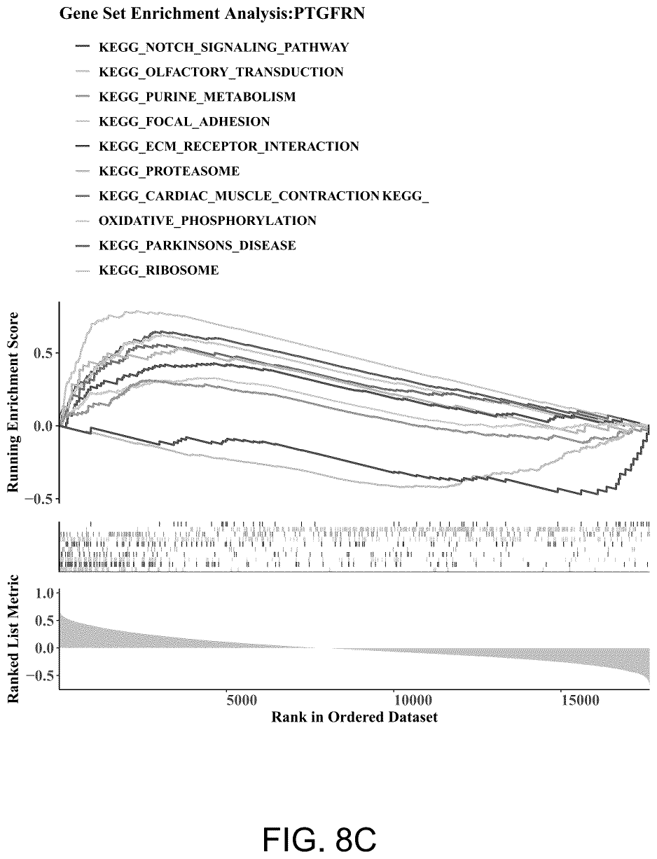

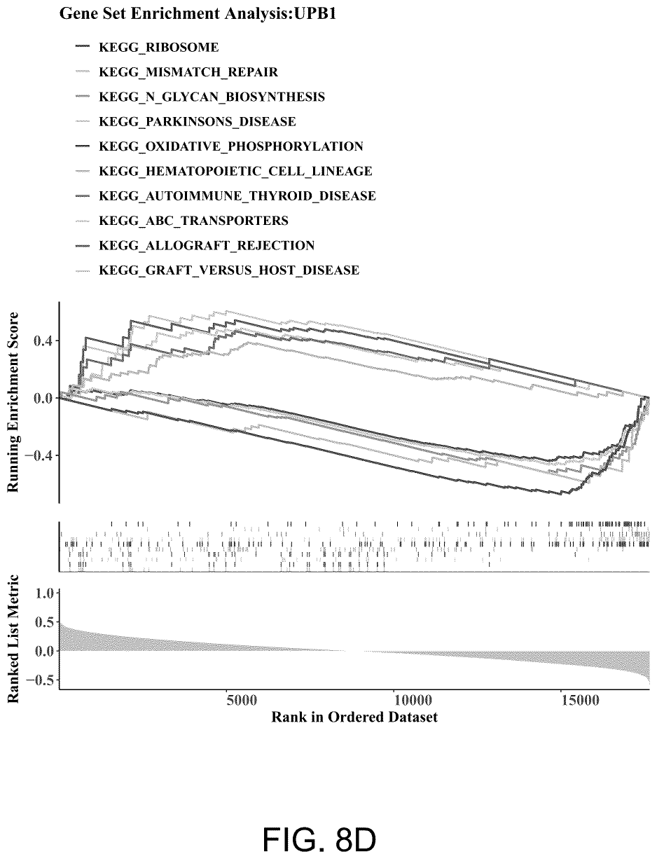

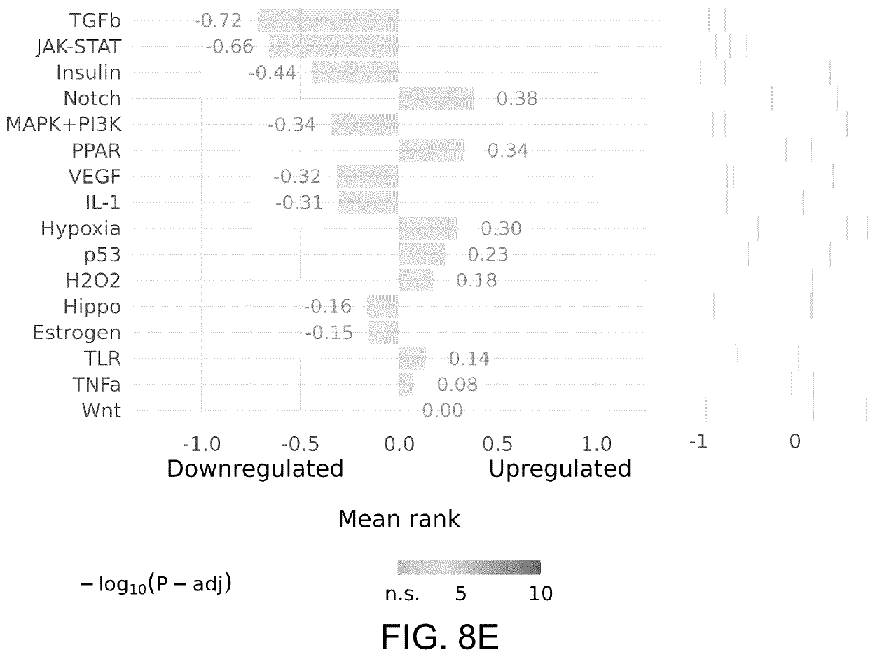

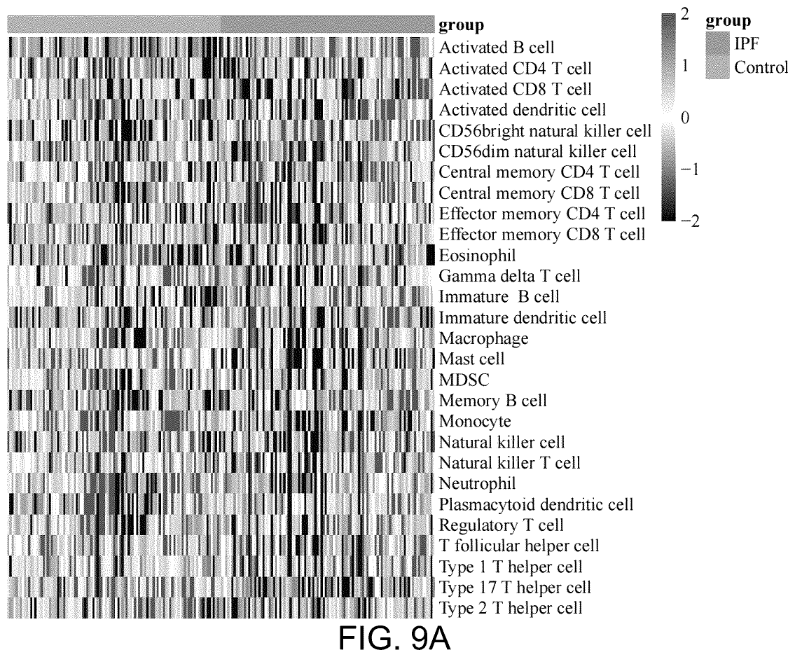

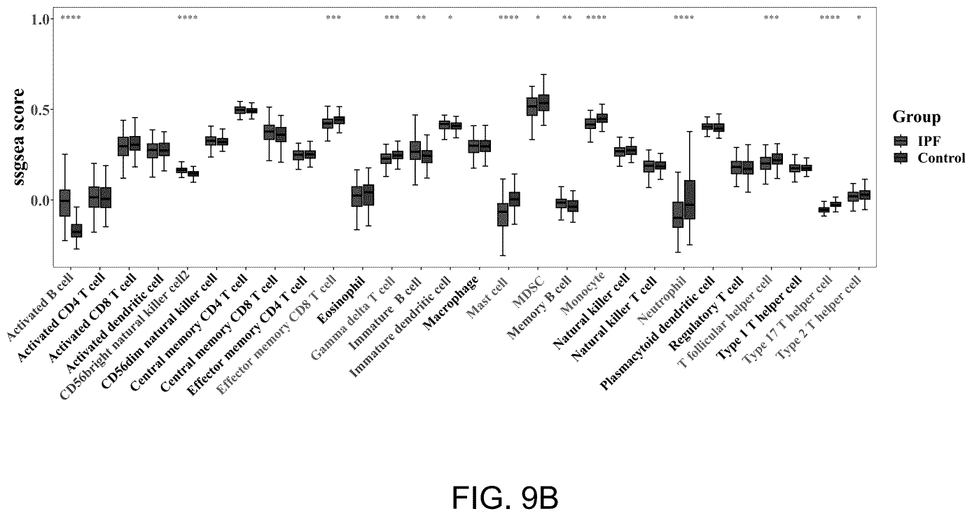

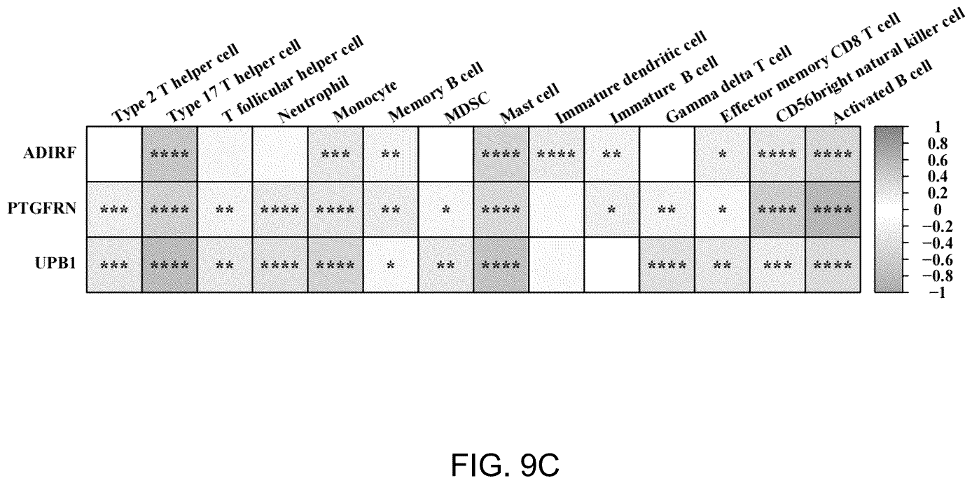

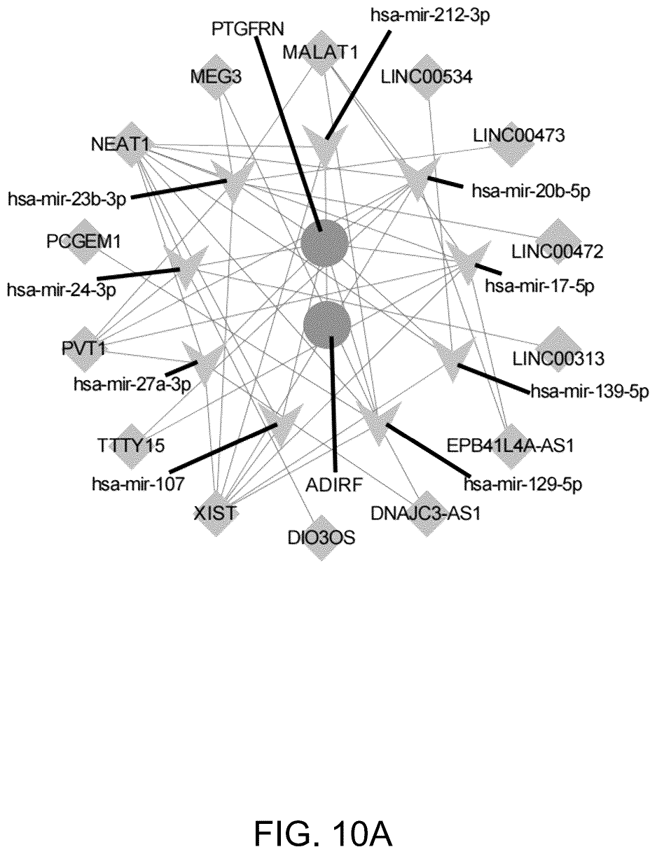

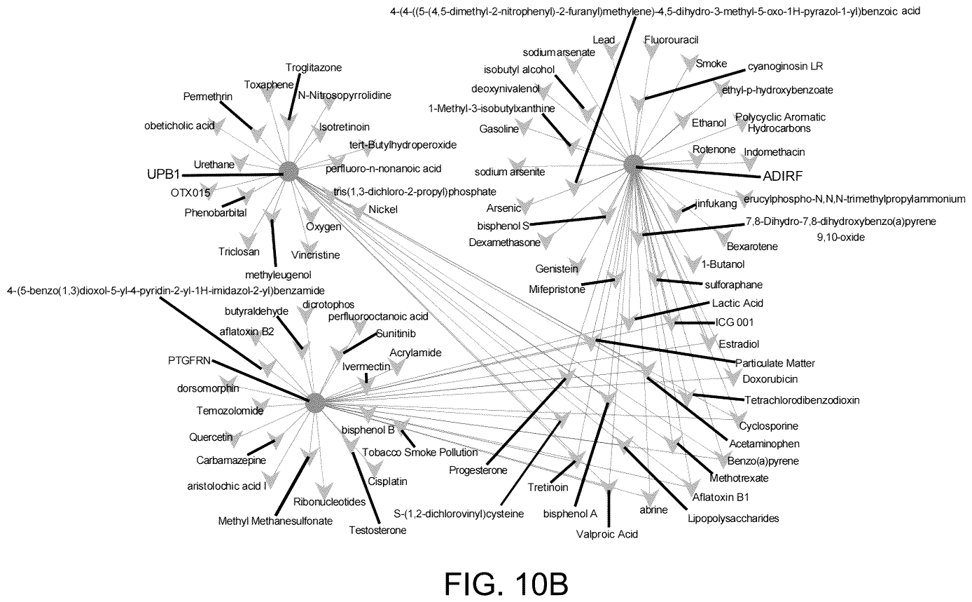

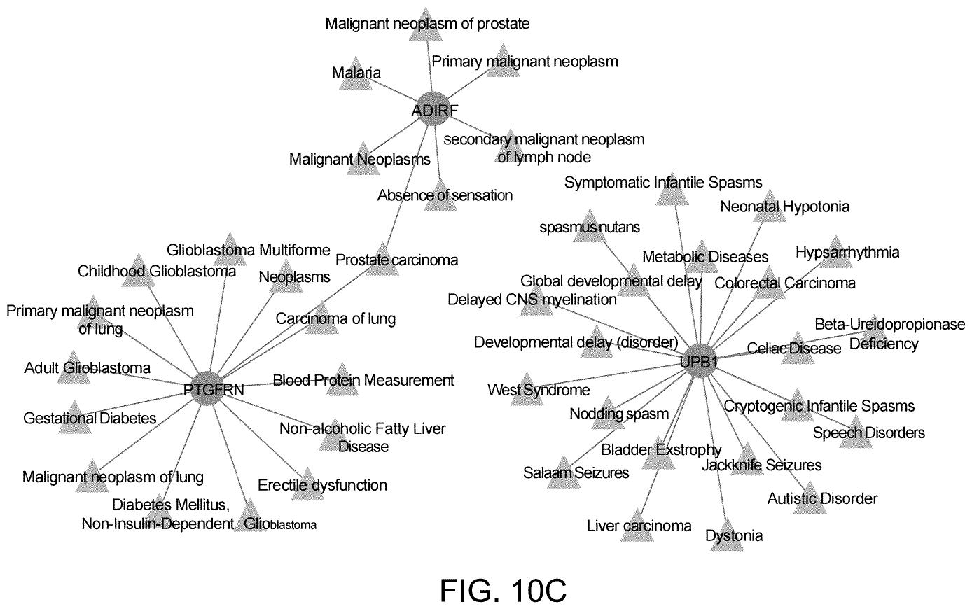

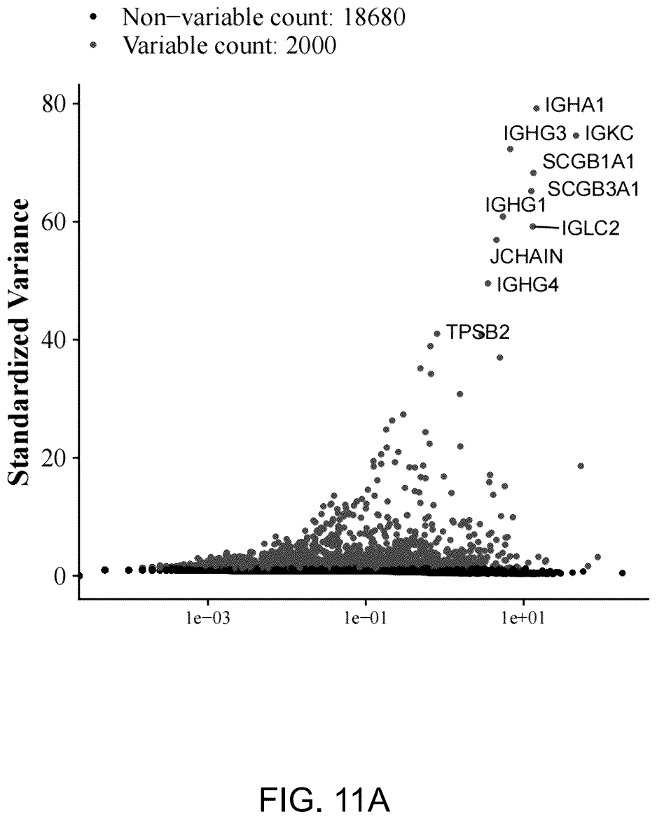

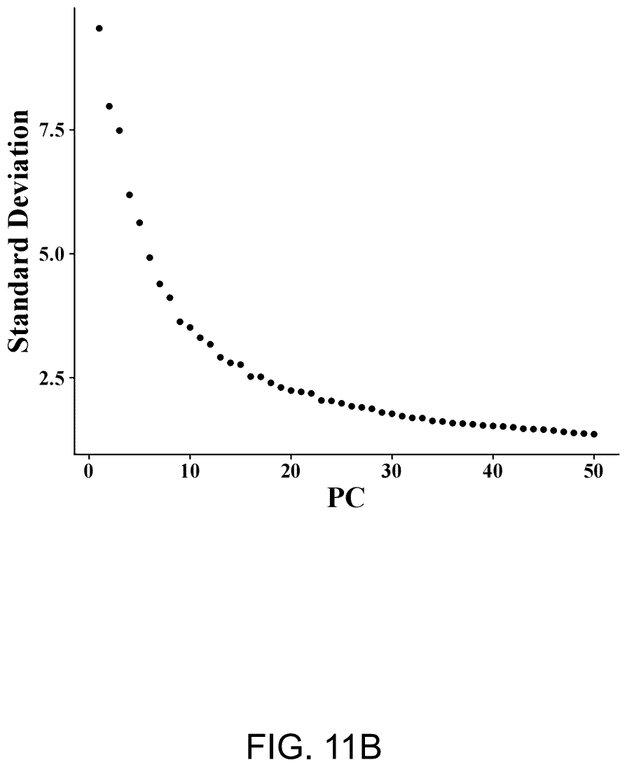

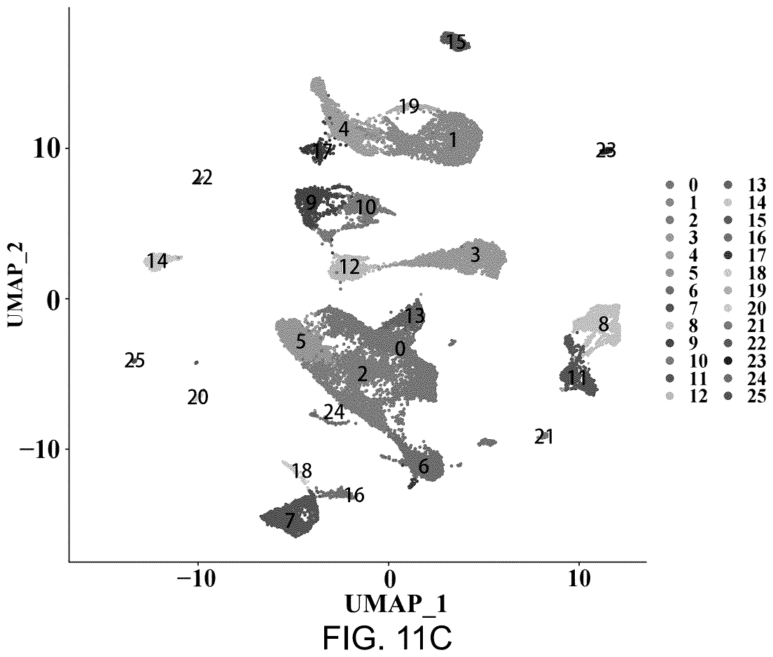

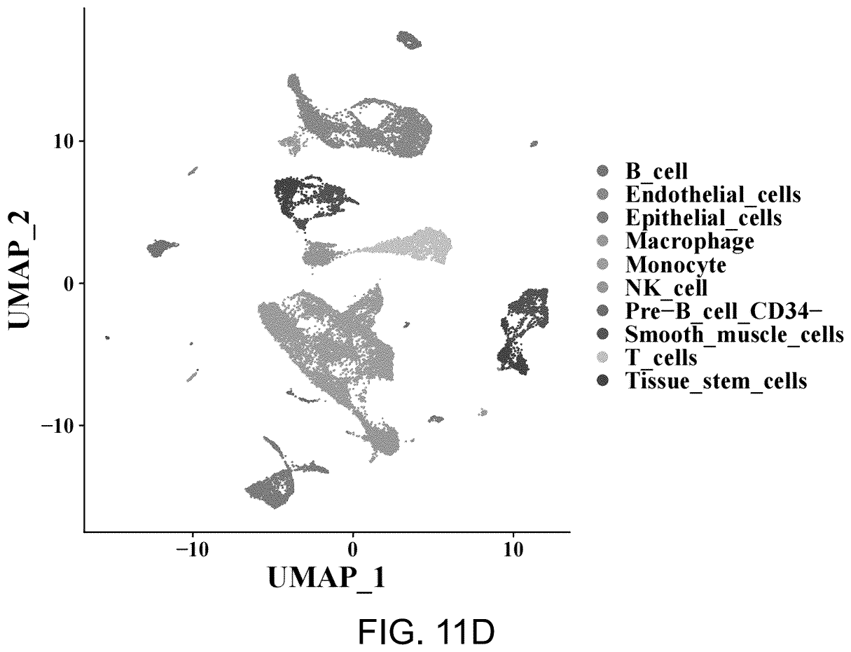

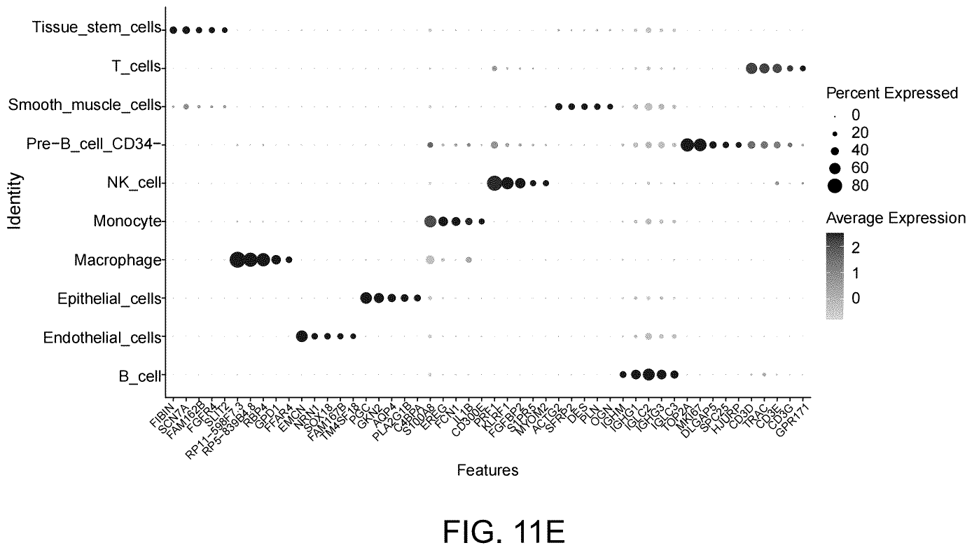

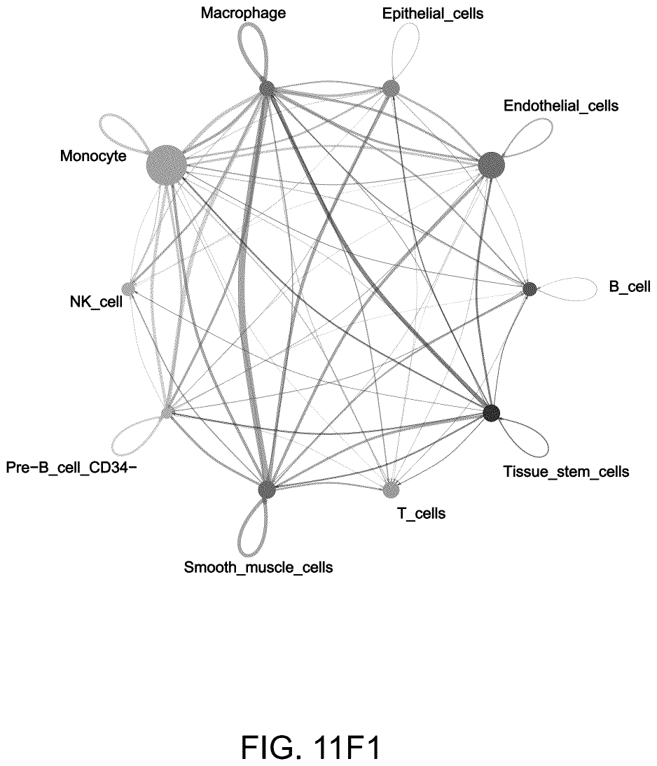

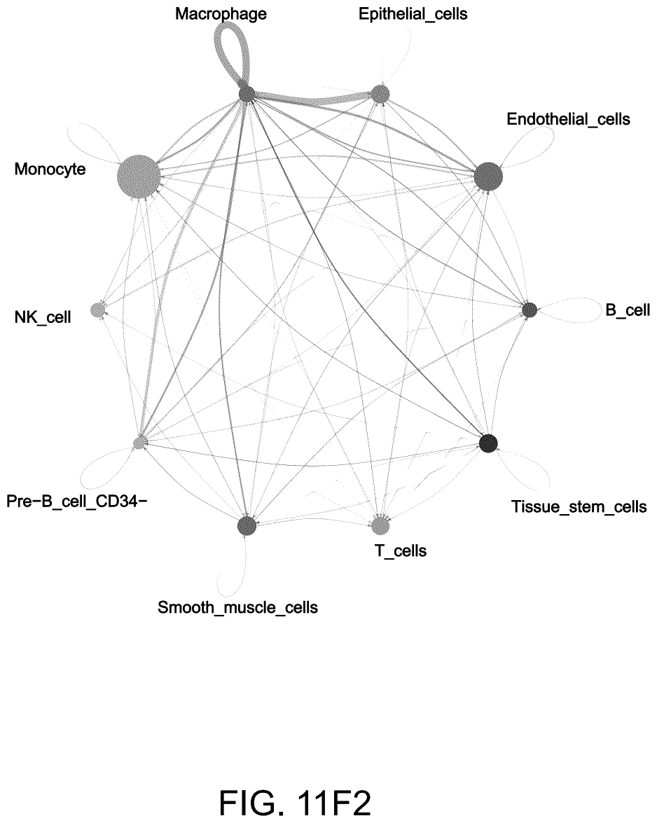

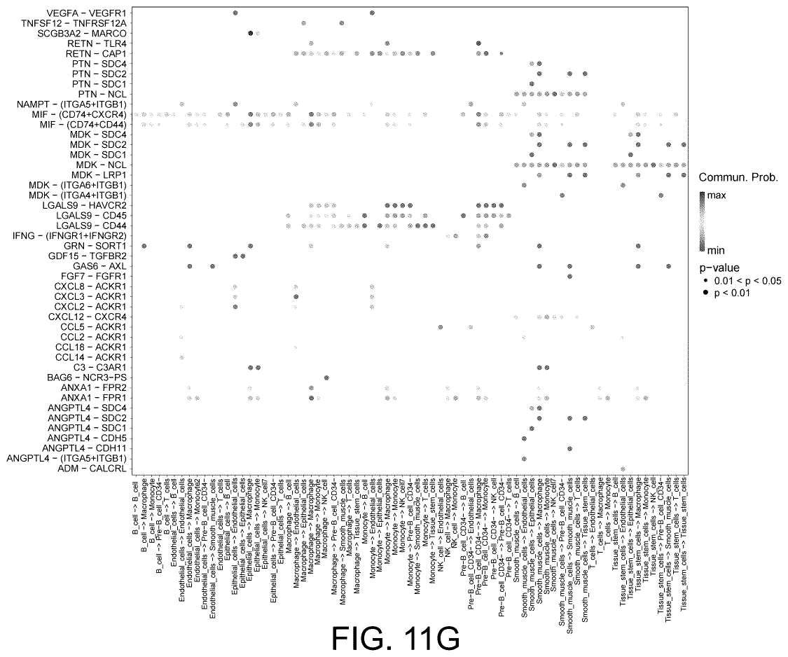

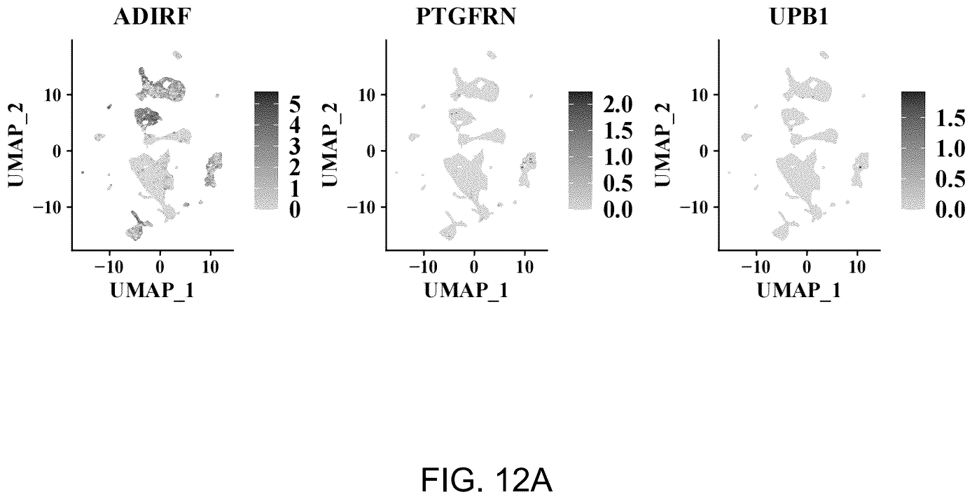

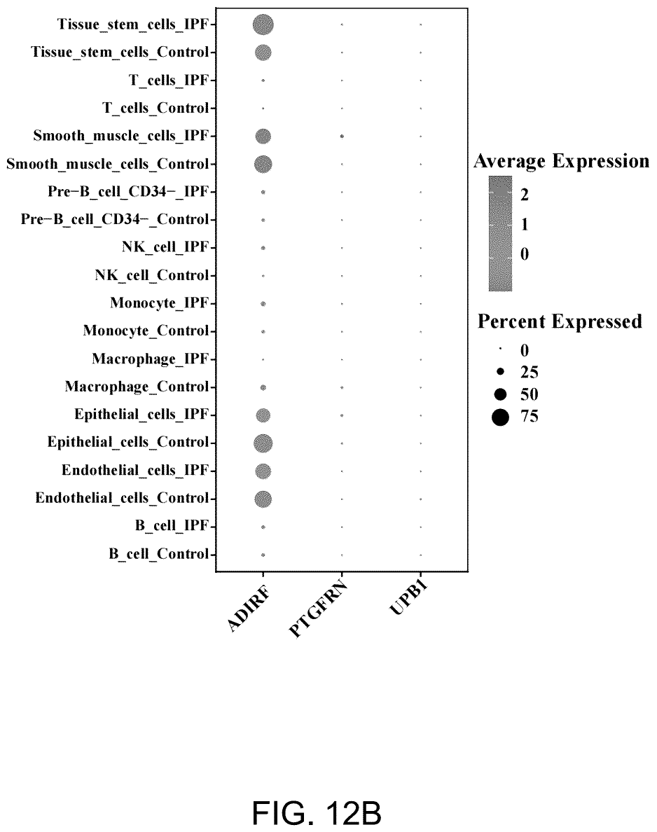

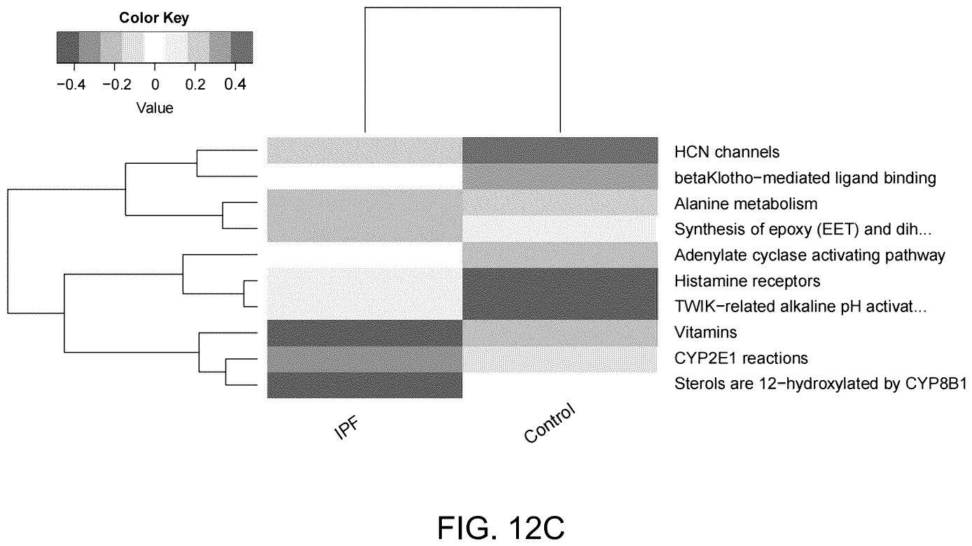

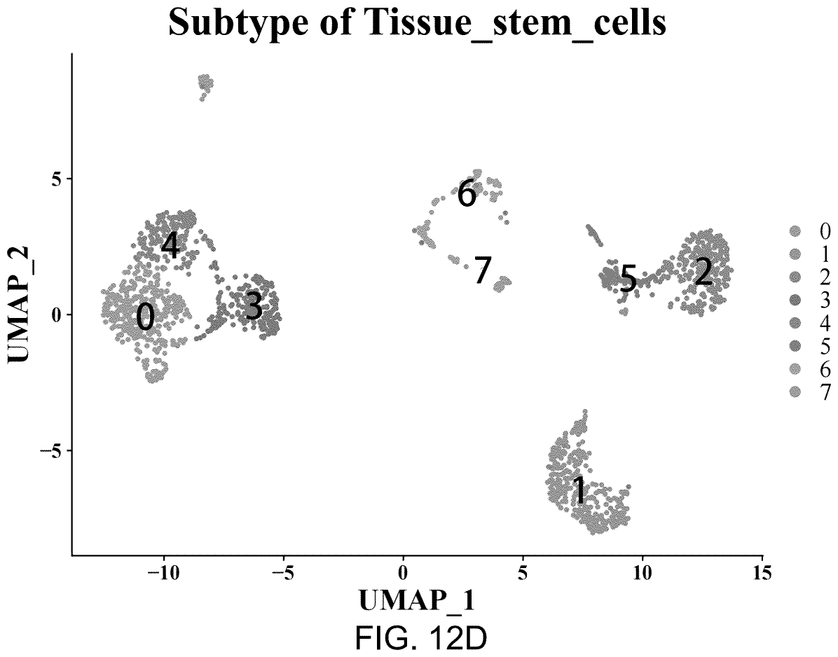

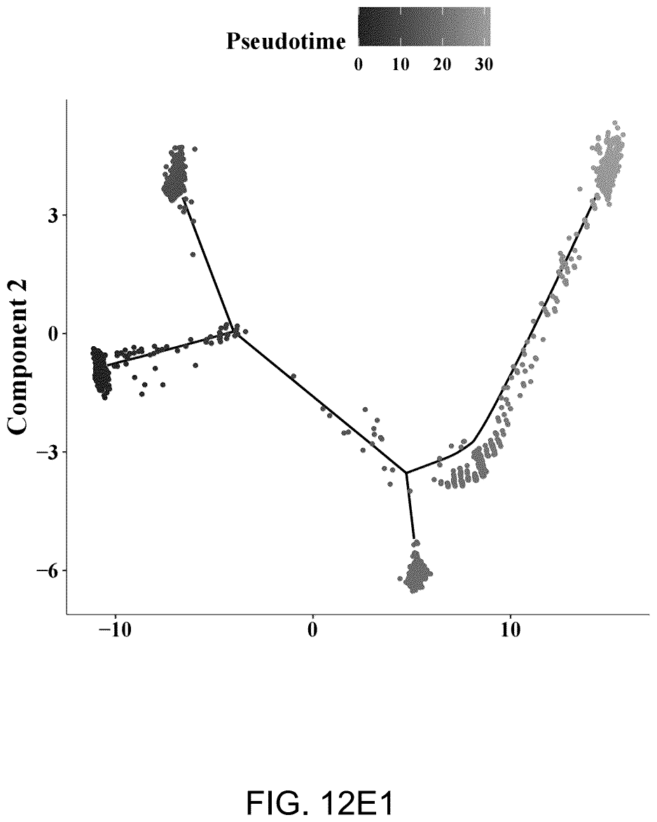

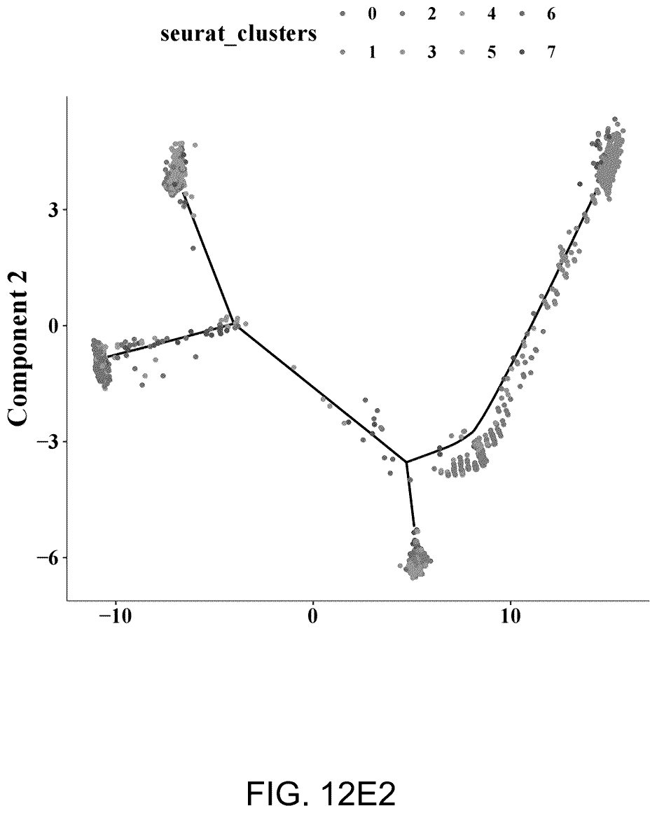

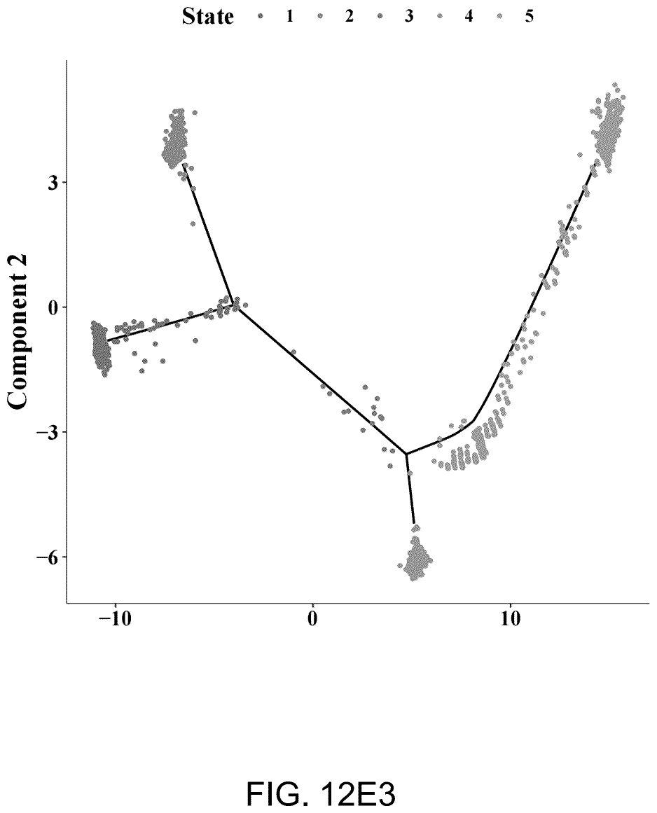

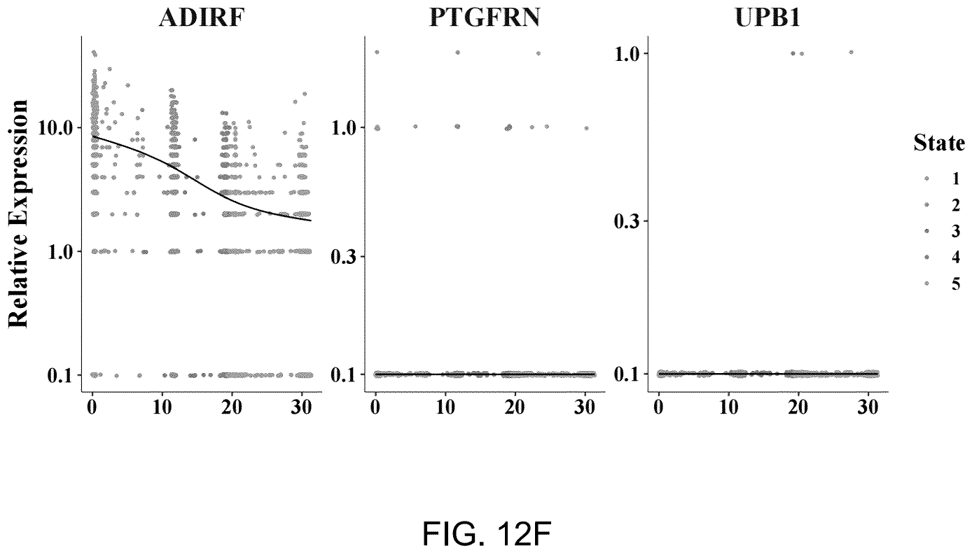

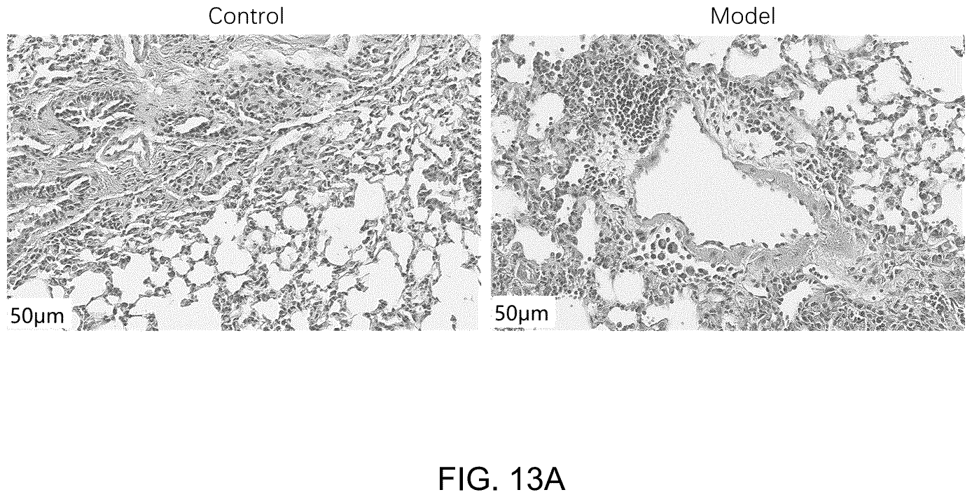

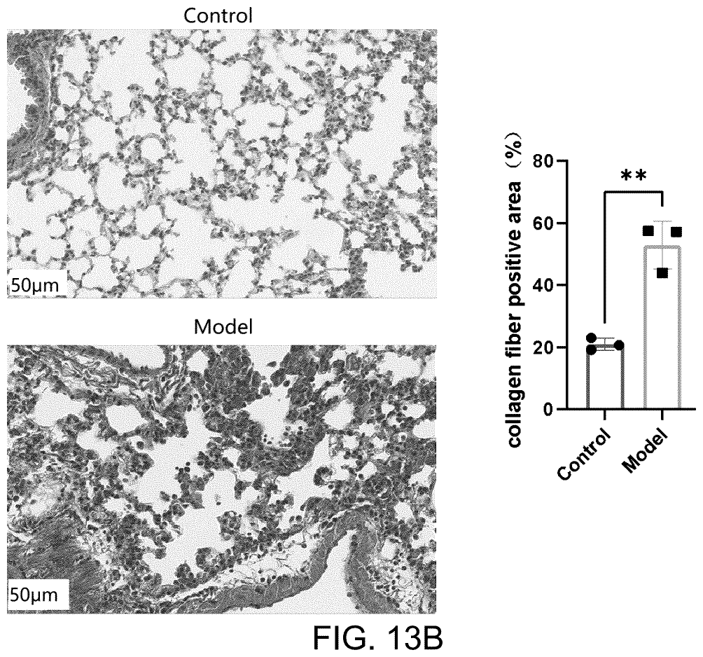

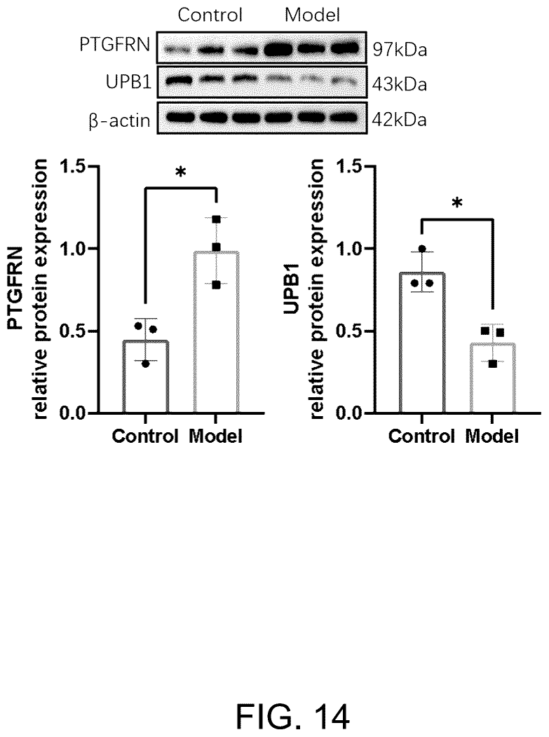

FIGS. 3J1-3J2: COL5A2-IPF;

FIG. 3K: CLEC12B-IPF.

FIG. 4A shows cross-validation of adjusted parameters in the LASSO analysis.

FIG. 4B shows the coefficient profile of 17 LASSO-selected genes.

FIG. 4C shows predictive accuracy with different numbers of feature genes.

FIG. 4D is a Venn diagram of relevant feature genes.

FIG. 4E and FIG. 4F are boxplots of candidate gene expression in the training and validation sets, respectively.

FIG. 4G and FIG. 4H show ROC curves of key genes in the training and validation sets, respectively.

FIGS. 5A1-5A3 are scatter plots of the correlation between candidate genes and IPF.

FIGS. 5B1-5B3 are forest plots of candidate genes and IPF.

FIGS. 5C1-5C3 are funnel plots of candidate genes and IPF.

FIG. 6A shows the construction of a nomogram model using key genes.

FIG. 6B shows the calibration curve of the nomogram model.

FIG. 6C shows the ROC curve of the nomogram model.

FIG. 6D shows the DCA (Decision Curve Analysis) of the nomogram model.

FIG. 7A and FIG. 7B show the visualization of the artificial neural network model in the training and validation sets, respectively.

FIG. 7C and FIG. 7D show the ROC curves of the neural network model in the training and validation sets, respectively.

FIG. 8A shows the GeneMANIA interaction network.

FIGS. 8B-8D show KEGG pathway enrichment maps for ADIRF, PTGFRN, and UPB1, respectively.

FIG. 8E shows the pathway activity analysis of key genes.

FIG. 9A shows a heatmap of immune cell infiltration scores between disease and control groups.

FIG. 9B is a boxplot of 28 immune infiltrating cells between the high and control groups.

FIG. 9C shows a heatmap of the correlation between key genes and differential immune cells.

FIG. 10A shows the mRNA-miRNA-lncRNA regulatory network.

FIG. 10B shows a network diagram of prognostic targets and predicted drugs.

FIG. 10C shows a network diagram of key genes and related diseases.

FIG. 11A shows the screening of highly variable genes.

FIG. 11B shows the scree plot of principal components.

FIG. 11C shows UMAP dimensionality reduction clustering.

FIG. 11D shows annotation of cell types.

FIG. 11E is a bubble chart of marker gene expression in different cell types.

FIGS. 11F1-11F2 shows intercellular communication among different cell types.

FIG. 11G is a bubble chart showing ligands and receptors of each cell type.

FIG. 12A shows the UMAP plot of key gene expression.

FIG. 12B is a bubble chart of key gene expression across cell types.

FIG. 12C shows pathway enrichment of epithelial cells between IPF and control samples.

FIG. 12D shows subcluster classification of critical cell types.

FIGS. 12E1-12E3 shows pseudotime trajectory analysis of key cell types.

FIG. 12F shows the expression trends of key genes across developmental stages in key cell types.

FIG. 13A shows the hematoxylin and eosin (H&E) staining.

FIG. 13B shows the Masson's trichrome staining.

FIG. 14 shows changes in PTGFRN and UPB1 protein levels in lung tissues from the normal and model groups.

### DESCRIPTION OF THE EMBODIMENTS

The following embodiment provides a further explanation of the present invention. In instances where specific technologies or conditions are not indicated, the methods or conditions may be those described in the literature of the field or as per the product instructions. Reagents or instruments not specifying the manufacturer's name are standard products available through regular commercial channels.

### 1. Materials and Methods

### 1.1 Data Collection

The Gene Expression Omnibus (GEO) database (http://www.ncbi.nlm.nih.gov/geo/) provides three publicly available datasets associated with IPF. The samples used in these datasets are derived from lung tissues. Specifically: The GSE150910 dataset (GPL24676 platform) includes 103 IPF samples and 103 normal samples. The GSE110147 dataset (GPL6244 platform) includes 22 IPF samples and 11 normal samples. Additionally, the GSE132771 dataset (GPL24676 platform) includes lung tissue data from 3 IPF samples and 3 normal samples, which were used for single-cell RNA sequencing (scRNA-seq) analysis. Furthermore, a literature search (PMID: 36611187) was conducted to identify 32 splicing factor-related genes (SF-RGs) associated with IPF, including the following genes: SRSF1, SRSF2, SRSF3, SRSF4, SRSF5, SRSF6, SRSF7, SRSF8, SRSF9, SRSF10, SRSF11, SRSF12, HNRNPA0, HNRNPA1, HNRNPA2B1, HNRNPA3, hnrnnpc, HNRNPCL1, hnrnnpd, HNRNPDL, HNRNPF, HNRNPH1, HNRNPL, HNRNPM, HNRNPR, HNRNPU, HNRNPUL1, and HNRNPUL2.

To explore the genetic basis of Idiopathic Pulmonary Fibrosis (IPF), summary data from the Genome-Wide Association Studies (GWAS) of IPF were collected from the Integrated Epidemiology Unit (IEU) Open GWAS database (https://gwas.mrcieu.ac.uk/). The data included 198,014 European samples, comprising 1,028 cases and 196,986 controls, with 16,380,413 SNPs. The relevant dataset for IPF (fin-b-IPF) was utilized for subsequent analysis.

### 1.2 Weighted Gene Co-Expression Network Analysis (WGCNA)

For the GSE150910 dataset, which contains all samples, a background gene set of 32 splicing factor-related genes (SF-RGs) was used. The single-sample Gene Set Enrichment Analysis (ssGSEA) method, implemented through the GSVA-package (v1.42.0), was used to calculate SF-RG scores. Differences between the IPF and normal samples were assessed using the Wilcoxon test.

Subsequently, WGCNA was performed using the WGCNA-package (v1.70.3) to identify gene modules associated with SF-RG scores. Outliers were identified and removed through clustering analysis. An optimal soft thresholding power (B) was selected within the range of 1 to 20 based on the scale-free network evaluation coefficient R2 exceeding the threshold of 0.85 and the average connectivity converging to 0. Genes within each module were grouped and color-coded, and Pearson correlation coefficients between gene modules and SF-RG scores were calculated. Modules with the highest positive or negative correlation were considered key modules (P<0.05), and genes in these modules were considered key genes.

### 1.3 Differential Expression Analysis and Functional Enrichment

Differential expression analysis of the gene expression matrix from the GSE150910 dataset was performed using DESeq2-package (v1.34.0) with P.adjust<0.05 and |log 2FoldChange (FC)|>1 as the selection criteria to identify differentially expressed genes (DEGs) between IPF and normal samples. Volcano plots and heatmaps of DEGs were generated using ggplot2-package (v3.4.1) and ComplexHeatmap-package (v2.14.0).

VennDiagram-package (v1.7.1) was used to intersect DEGs and key module genes to identify candidate genes for further investigation. Functional enrichment analysis for Gene Ontology (GO) and Kyoto Encyclopedia of Genes and Genomes (KEGG) pathways was conducted using clusterProfiler-package (v4.2.2), focusing on biological processes (BP), cellular components (CCs), molecular functions (MFs), and signaling pathways. A p-value of <0.05 was considered significant.

### 1.4 MR Analysis Data Preprocessing

Mendelian randomization (MR) analysis was employed to explore causal relationships between the identified candidate genes (exposure factors) and IPF (outcome). Expression Quantitative Trait Loci (eQTL) GWAS data for the candidate genes were obtained from the IEU Open GWAS database for subsequent analysis.

It is important to note that the MR design must adhere to three key assumptions:

Association Assumption: The genetic variations used as instrumental variables (IVs) must be strongly associated with the exposure factor.

Independence Assumption: IVs must be independent of any confounding factors that may affect the relationship between the exposure and the outcome.

Exclusion Restriction Assumption: IVs affect the outcome solely through their impact on the exposure, with no alternative pathways.

These assumptions guide the quality control (QC) process. The TwoSampleMR-package (v0.5.6) was used to extract relevant IVs (SNPs) associated with the exposure factor (P<5× 10\hat{ }-8). SNPs with linkage disequilibrium (LD) (r2=0.001, kb=10) were excluded. IVs significantly associated with the outcome were removed, and the harmonise_data function was used to align effect alleles and effect sizes for accuracy. Finally, candidate genes with at least three SNPs were selected as exposure factors for subsequent MR analysis.

### 1.5 MR Analysis and Sensitivity Analysis

MR analysis was conducted using five different algorithms: MR Egger, weighted median, inverse variance weighting (IVW), simple mode, and weighted mode. The odds ratio (OR) and p-values were calculated for the causal relationship between candidate genes and IPF, with a primary focus on the IVW results.

Sensitivity analysis was performed in multiple steps to evaluate the robustness of the MR estimates. Cochran's Q-test and MR-Egger intercept test were used to assess heterogeneity among IVs and horizontal pleiotropy, respectively. Exposure factors that did not pass heterogeneity and pleiotropy tests (P<0.05) were excluded.

Additionally, leave-one-out (LOO) analysis was conducted to assess whether any individual SNP significantly influenced the MR estimates.

Finally, Steiger's test was used to verify the directionality of causal relationships. Exposure factors with P<0.05 and correct_causal_direction=TRUE were considered for follow-up analysis as key genes.

### 1.6 Machine Learning Algorithms

In the GSE150910 dataset, feature genes were identified by integrating Least Absolute Shrinkage and Selection Operator (LASSO) regression and Support Vector Machine-Recursive Feature Elimination (SVM-RFE) algorithms. LASSO analysis was performed using the glmnet package (v4.1-4) (PMID: 20808728), which introduces a penalization function to reduce data dimensionality, thereby mitigating multicollinearity and overfitting in multivariate regression models. The SVM-RFE algorithm was implemented using the caret package (v6.0-93), which identifies the optimal subset of variables by recursively eliminating features based on weight vector rankings. Feature genes derived from both LASSO and SVM-RFE analyses were intersected, and the overlapping genes were designated as biomarkers for this study.

Differential expression of the identified biomarkers between the IPF and control groups was assessed in the GSE150910 and GSE110147 datasets using the Wilcoxon rank-sum test. Receiver Operating Characteristic (ROC) curves were generated using the pROC package (v1.18.0) (PMID: 21414208) to evaluate the diagnostic performance of each biomarker in distinguishing IPF from normal samples. An area under the curve (AUC) value exceeding 0.70 was considered indicative of satisfactory diagnostic utility.

### 1.7 Nomogram Construction

A diagnostic nomogram model was constructed based on the expression profiles of the identified biomarkers to facilitate accurate clinical prediction of IPF. The rms package (v6.5-1) (PMID: 32530136) was used to generate the nomogram based on GSE150910 expression data. Model performance was evaluated through calibration plots and ROC curves, while clinical utility was assessed via Decision Curve Analysis (DCA).

### 1.8 Artificial Neural Network Model

Artificial Neural Networks (ANNs), composed of interconnected layers of nodes (neurons or units), were utilized to build a diagnostic model. The network weights between neurons were iteratively adjusted to minimize discrepancies between predicted and observed outcomes. In this study, an ANN diagnostic model was constructed based on biomarker expression profiles using the neuralnet package (v1.5.3) (PMID: 30505247), applied to both GSE150910 and GSE110147 datasets. ROC curves were plotted to evaluate the model's performance in diagnosing IPF.

### 1.9 Biomarker Enrichment Analysis

The identified biomarkers were uploaded to the GeneMANIA database (http://genemania.org) to identify functionally related genes and associated biological pathways. To gain further insights into the functional roles of these biomarkers in IPF pathogenesis, Spearman correlation coefficients between the biomarkers and all other genes were calculated within the GSE150910 dataset. Genes were ranked by correlation strength, and Gene Set Enrichment Analysis (GSEA) was performed using the clusterProfiler package, referencing the KEGG subset of the Molecular Signatures Database (“c2.cp.kegg.v2023.1.Hs.symbols” from MSigDB, http://www.broadinstitute.org/gsea/msigdb/index.jsp). The false discovery rate (FDR) threshold was set at <0.05. In addition, the SPEED2 database (https://speed2.sys-bio.net/) was queried to identify upstream signaling pathway activities associated with the biomarkers.

### 1.10 Immune Infiltration Analysis

The single-sample Gene Set Enrichment Analysis (ssGSEA) algorithm, implemented via the GSVA package, was used to quantify the infiltration scores of 28 immune cell types in each sample within the GSE150910 dataset. Wilcoxon rank-sum testing was applied to compare immune infiltration levels between IPF and control groups. Spearman correlations between biomarker expression and significantly altered immune cell types were further evaluated using the psych package (v2.1.6).

### 1.11 Regulatory Network Analysis and Drug Prediction

Regulatory microRNAs (miRNAs) targeting the identified biomarkers were retrieved from the miRNet (https://www.mirnet.ca/) and NetworkAnalyst (https://www.networkanalyst.ca/) platforms. Upstream long non-coding RNAs (lncRNAs) of these miRNAs were collected from miRcode (http://www.mircode.org/) and StarBase (http://starbase.sysu.edu.cn/) databases. The resulting mRNA-miRNA-lncRNA regulatory network was visualized using Cytoscape (v3.8.2) (PMID: 14597658).

In addition, the Comparative Toxicogenomics Database (CTD, http://ctdbase.org/) was queried to identify potential therapeutic agents targeting the biomarkers, aiming to explore candidate compounds for IPF treatment. Associated diseases were also predicted using the DisGeNET database (https://www.disgenet.org/) based on biomarker-related gene-disease associations.

### 1.12 Single-Cell Analysis

To further investigate IPF at the single-cell level, single-cell RNA sequencing (scRNA-seq) analysis was performed on the GSE132771 dataset using the Seurat package (v4.1.0) (PMID: 25605792). Cells were filtered based on the following quality control criteria: (1) 400-3000 detected genes per cell; (2) fewer than 15,000 total molecular counts per cell; and (3) mitochondrial gene content less than 5%. Data normalization and identification of highly variable genes were performed using the NormalizeData and FindVariableFeatures functions, respectively, with the top 2,000 most variable genes retained for downstream analysis.

Principal component analysis (PCA) was conducted, and statistically significant principal components (PCs) were selected using the JackStrawPlot function (P<0.05). Unsupervised clustering was then performed using the FindNeighbors and FindClusters functions (resolution=0.4), and clusters were visualized via Uniform Manifold Approximation and Projection (UMAP). Cell type annotation was conducted using the SingleR package (v2.0.0) (PMID: 30643263), and cluster-specific marker genes were identified using the FindAllMarkers function.

Expression levels of the identified biomarkers were compared across cell clusters, and the clusters with high expression were designated as key cell populations. Functional enrichment analysis of key cells in IPF versus control samples was conducted using the ReactomeGSA package (v1.12.0) (PMID: 32907876). Intercellular communication networks were evaluated using the CellChat package (v1.5.0) (PMID: 37827697). Subsequently, dimensionality reduction was applied to key cell populations to delineate cellular subtypes, and pseudotime trajectory analysis was performed using the Monocle package (v2.22.0) (PMID: 24658644).

### 1.13 In Vivo Validation of Biomarkers

### 1.13.1 Experimental Animals and Model Establishment

A total of 24 specific pathogen-free (SPF) male C57BL/6 mice (8 weeks old) were used in this study. Mice were housed in a well-ventilated environment with controlled humidity, free access to sterile food and water, and a 12-hour light/dark cycle. After acclimation, the animals were randomly divided into two groups: control group (n=12) and model group (n=12). Pulmonary fibrosis was induced in the model group by intratracheal administration of bleomycin (3 mg/kg in 0.9% saline) on Day 1. Control mice received an equal volume of 0.9% saline. On Day 28 after bleomycin administration, mice were anesthetized with 2% isoflurane inhalation, and lung tissues were collected for further analysis.

### 1.13.2 Tissue Processing and Sectioning

Lung tissues were fixed in 4% paraformaldehyde for 24 hours, dehydrated through graded ethanol, and embedded in paraffin. Paraffin sections (4-5 μm thick) were prepared for H&E staining, Masson's trichrome staining, and immunofluorescence analysis. H&E staining was used to assess alveolitis, while Masson staining was used to evaluate the extent of pulmonary fibrosis.

### 1.13.3 Western Blot Analysis of PTGFRN and UPB1 Protein Levels

Western blotting was performed using standard SDS-polyacrylamide gel electrophoresis (SDS-PAGE) protocols. Total protein was extracted from lung tissues, separated by SDS-PAGE, and transferred to polyvinylidene difluoride (PVDF) membranes. Membranes were incubated overnight at 4 °C with primary antibodies against PTGFRN, UPB1, and β-actin, followed by incubation with horseradish peroxidase (HRP)-conjugated secondary antibodies (1:5000 dilution) for 1 hour at room temperature. Signal detection was performed using the SuperSignal West Dura chemiluminescent substrate, and band intensities were quantified using ImageJ software. The relative expression of target proteins was calculated as:

Relative expression=(Target protein optical density/β-actin optical density)×10ⁿ, and results were expressed as mean±standard deviation.

### 1.14 Statistical Analysis

All bioinformatic and statistical analyses were conducted using R software (v4.1.0). The Wilcoxon rank-sum test was used to compare scores between two groups. A P value<0.05 was considered statistically significant.

### 2. Results

### 2.1 Identification of 1,217 Key Module Genes via WGCNA

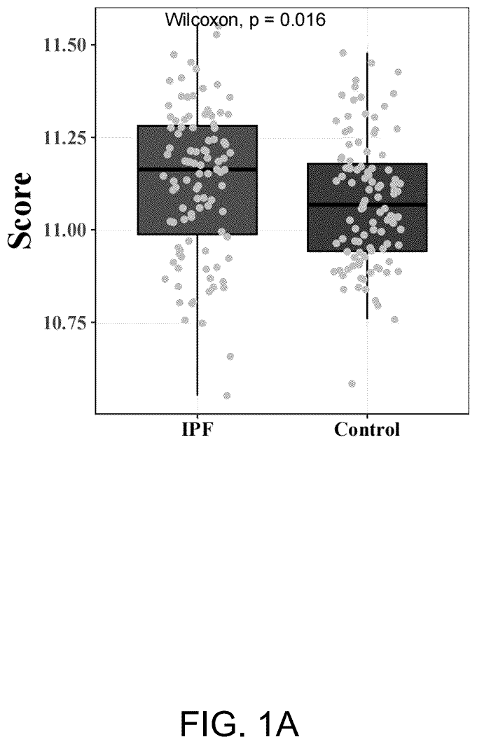

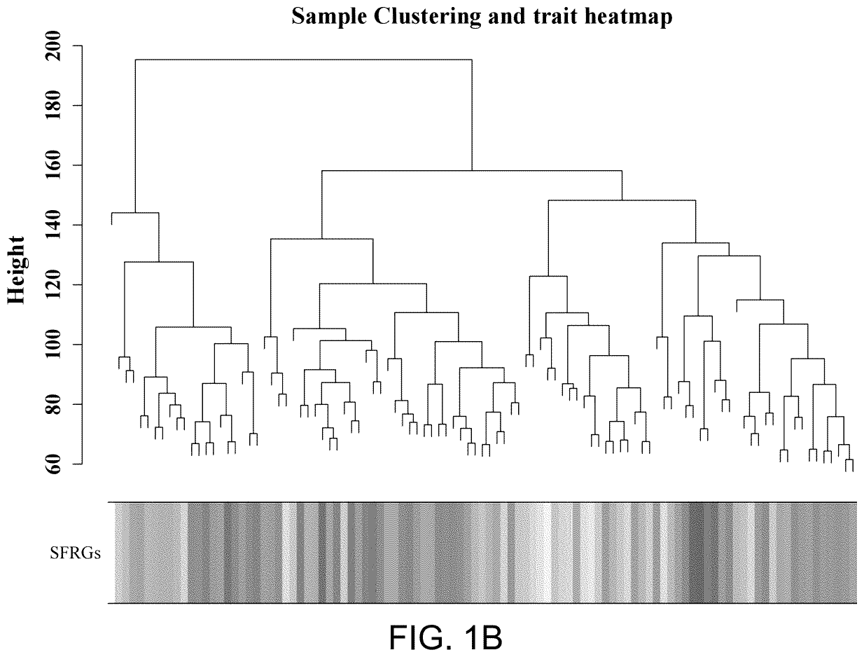

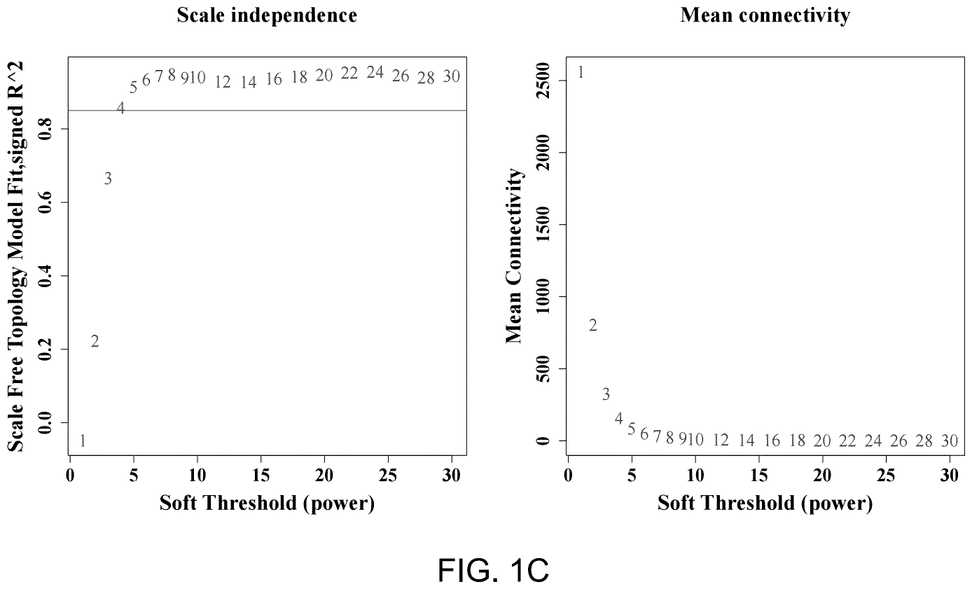

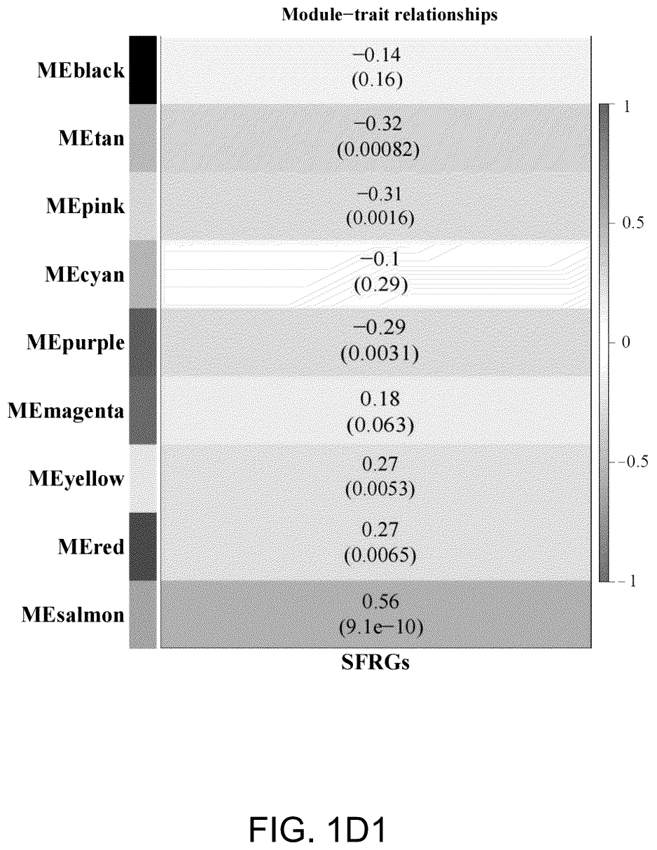

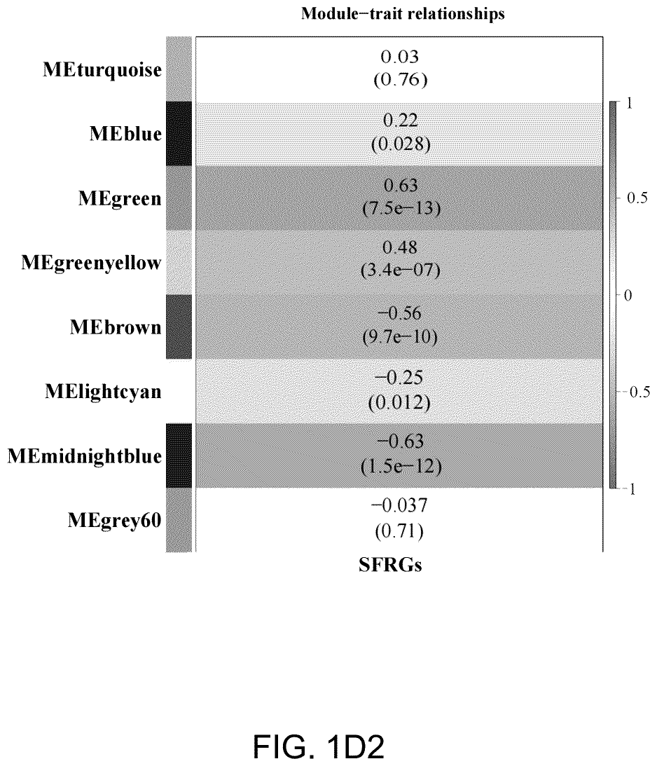

Weighted gene co-expression network analysis (WGCNA) identified a total of 1,217 key module genes. The ssGSEA algorithm demonstrated a significant difference in SF-RGs scores between idiopathic pulmonary fibrosis (IPF) and normal samples in the GSE150910 dataset (P=0.016), with higher SF-RGs scores observed in IPF samples (FIG. 1A). No outlier samples were detected by hierarchical clustering (FIG. 1B). A scale-free network was constructed with a soft-thresholding power (β) of 7 (R²=0.85; FIG. 1C).

Seventeen co-expression modules were identified (FIGS. 1D1-1D2), among which the MEgreen module showed the strongest positive correlation with SF-RGs scores (correlation=0.63, P<0.0001), and the MEmidnightblue module showed the strongest negative correlation (correlation=−0.63, P<0.0001). The MEgreen and MEmidnightblue modules contained 996 and 221 genes, respectively, yielding a total of 1,217 genes defined as SF-RGs-associated key module genes.

### 2.2 Functional Annotation of Candidate Genes

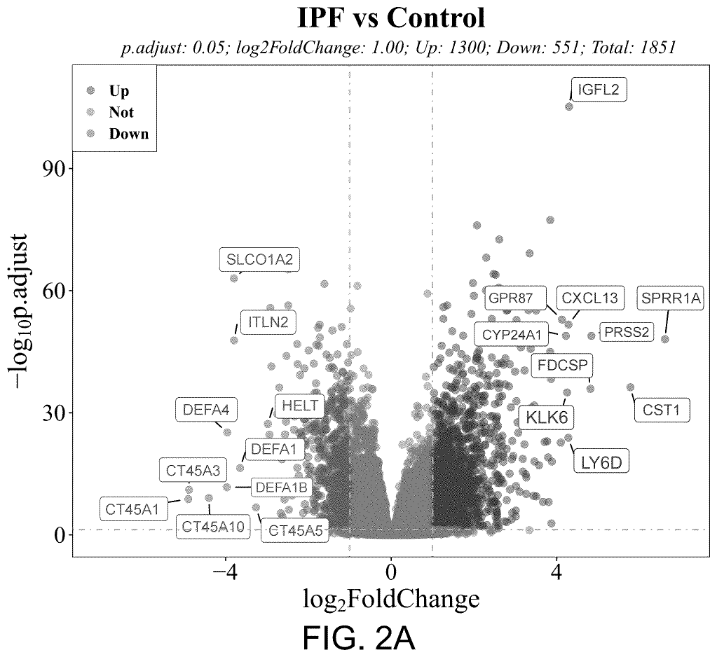

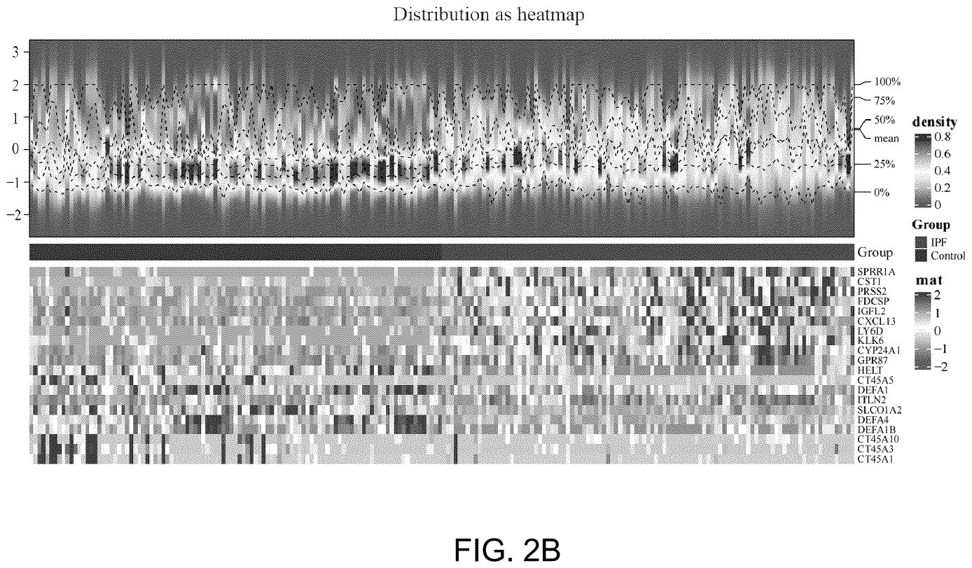

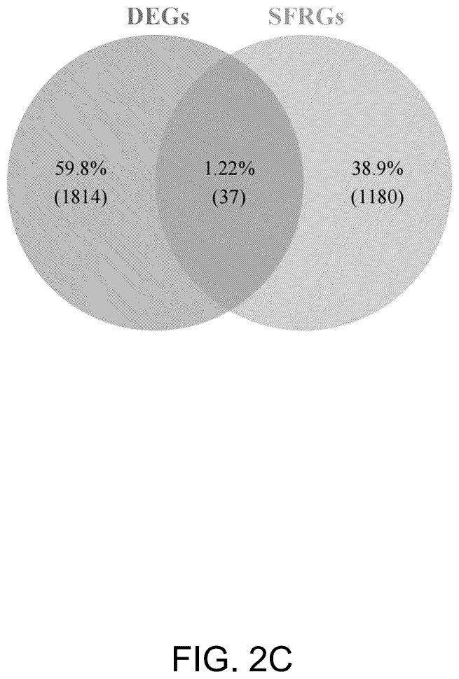

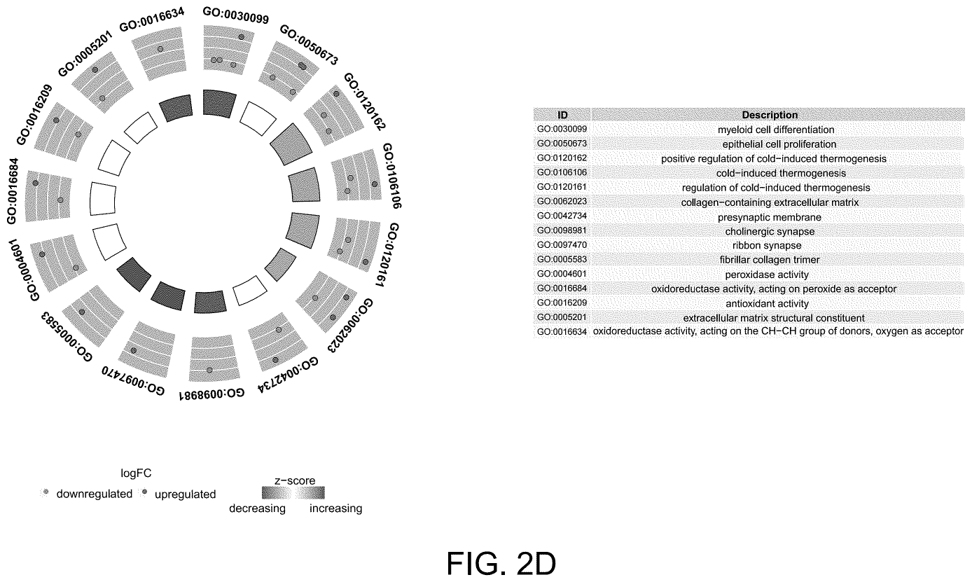

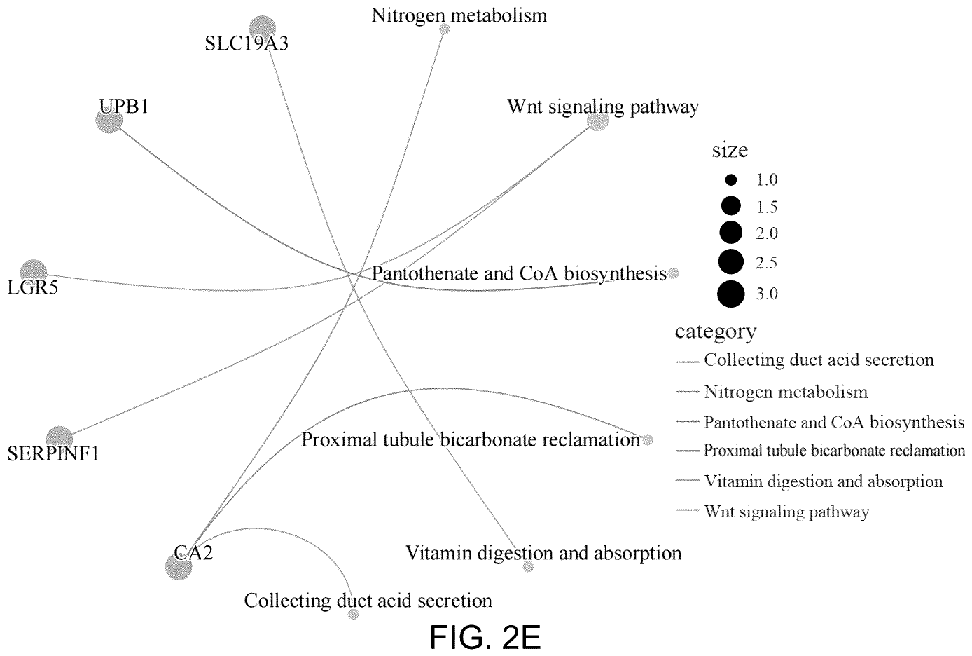

A total of 1,851 differentially expressed genes (DEGs) were identified between IPF and normal samples in the GSE150910 dataset, including 1,300 upregulated and 551 downregulated genes in the IPF group (FIGS. 2A-2B). Intersecting the 1,851 DEGs with the 1,217 key module genes resulted in 37 candidate genes (FIG. 2C).

Gene enrichment analysis of these 37 candidate genes identified 185 biological processes (BPs), 7 cellular components (CCs), 24 molecular functions (MFs), and 6 Kyoto Encyclopedia of Genes and Genomes (KEGG) pathways. In the GO-BP category, candidate genes were primarily involved in myeloid dendritic cell differentiation, epithelial cell proliferation, and cold-induced thermogenesis (FIG. 2D). Enriched CC terms included collagen-containing extracellular matrix, presynaptic membrane, and cholinergic synapse. In the MF category, the genes were significantly enriched in peroxidase activity, oxidoreductase activity, and antioxidant activity (FIG. 2D). KEGG pathway analysis revealed enrichment in pathways such as nitrogen metabolism, Wnt signaling pathway, pantothenate and CoA biosynthesis, and proximal tubule bicarbonate reclamation (FIG. 2E).

### 2.3 Causal Association Between Eight Key Genes and IPF

The 37 candidate genes were treated as exposure variables in a Mendelian randomization (MR) framework, with IPF as the outcome. The inverse-variance weighted (IVW) method identified 11 genes with a statistically significant causal association with IPF (P<0.05) (Table 1). No heterogeneity was detected via the Cochran's Q test (P>0.05) (Table 2).

However, potential horizontal pleiotropy was observed for CLEC12B and FAXDC2 in the MR-Egger intercept test, and these genes were subsequently excluded (Table 3). Leave-one-out (LOO) analysis showed no notable outliers among SNPs, supporting the robustness of the MR results (FIGS. 3A-3K).

Finally, only eight genes passed the Steiger directionality test (P<0.05, correct_causal_direction=TRUE) (Table 4), confirming a valid causal direction from gene expression to IPF. These genes—UPB1, COL5A2, GPR17, ADIRF, PTGFRN, MPP3, IRF4, and BATF2—were designated as key causal genes for subsequent analysis.

TABLE 1
MR Analysis Results of Significant Causal Exposure Factors Associated with IPF

| id.exposure | outcome | SYMBOL | nsnp | pval | or |
|---|---|---|---|---|---|
| eqtl-a-ENSG00000100024 | Idiopathic | UPB1 | 67 | 0.005222066 | 0.953537482 |
| eqtl-a-ENSG00000104267 | pulmonary | CA2 | 165 | 0.019854474 | 0.95514298 |
| eqtl-a-ENSG00000134247 | fibrosis ∥ | PTGFRN | 20 | 0.003450627 | 0.820933403 |
| eqtl-a-ENSG00000137265 | id:finn-b-IPF | IRF4 | 15 | 2.09E−07 | 0.53312005 |
| eqtl-a-ENSG00000144230 | | GPR17 | 8 | 0.020204653 | 0.586234386 |
| eqtl-a-ENSG00000148671 | | ADIRF | 21 | 0.008425074 | 1.298686864 |
| eqtl-a-ENSG00000161647 | | MPP3 | 3 | 0.028666918 | 2.216220753 |
| eqtl-a-ENSG00000168062 | | BATF2 | 11 | 0.000184138 | 1.871718924 |
| eqtl-a-ENSG00000170271 | | FAXDC2 | 100 | 0.001006157 | 0.945946138 |
| eqtl-a-ENSG00000204262 | | COL5A2 | 54 | 0.000162921 | 1.323155565 |
| eqtl-a-ENSG00000256660 | | CLEC12B | 73 | 7.64E−07 | 1.104711611 |

TABLE 2
Heterogeneity Test Results of Candidate Genes in MR Analysis for IPF

| id.exposure | outcome | SYMBOL | Q | Q_df | Q_pval |
|---|---|---|---|---|---|
| eqtl-a-ENSG00000100024 | Idiopathic | UPB1 | 69.50273447 | 66 | 0.360374666 |
| eqtl-a-ENSG00000104267 | pulmonary | CA2 | 67.71686587 | 164 | 1 |
| eqtl-a-ENSG00000134247 | fibrosis ∥ | PTGFRN | 15.52130014 | 19 | 0.688939264 |
| eqtl-a-ENSG00000137265 | id:finn-b-IPF | IRF4 | 7.853282375 | 14 | 0.896828682 |
| eqtl-a-ENSG00000144230 | | GPR17 | 2.270307779 | 7 | 0.943378548 |
| eqtl-a-ENSG00000148671 | | ADIRF | 6.882149133 | 20 | 0.997054023 |
| eqtl-a-ENSG00000161647 | | MPP3 | 0.443896389 | 2 | 0.800956857 |
| eqtl-a-ENSG00000168062 | | BATF2 | 15.56763077 | 10 | 0.112696542 |
| eqtl-a-ENSG00000170271 | | FAXDC2 | 66.90689151 | 99 | 0.994441519 |
| eqtl-a-ENSG00000204262 | | COL5A2 | 70.10761597 | 53 | 0.057782481 |
| eqtl-a-ENSG00000256660 | | CLEC12B | 75.50217655 | 72 | 0.365943304 |

TABLE 3
Horizontal Pleiotropy Test Results of Candidate Genes in MRAnalysis for IPF

| id.exposure | outcome | SYMBOL | egger_intercept | se | pval |
|---|---|---|---|---|---|
| eqtl-a-ENSG00000100024 | Idiopathic | UPB1 | −0.019394807 | 0.014756268 | 0.193349791 |
| eqtl-a-ENSG00000104267 | pulmonary | CA2 | 0.01023481 | 0.011693882 | 0.382737816 |
| eqtl-a-ENSG00000134247 | fibrosis ∥ | PTGFRN | −0.017565328 | 0.03100827 | 0.578068242 |
| eqtl-a-ENSG00000137265 | id:finn-b-IPF | IRF4 | 0.008344586 | 0.054835964 | 0.881386365 |
| eqtl-a-ENSG00000144230 | | GPR17 | 0.107927335 | 0.120995204 | 0.406742615 |
| eqtl-a-ENSG00000148671 | | ADIRF | 0.022663723 | 0.039751735 | 0.575268947 |
| eqtl-a-ENSG00000161647 | | MPP3 | −0.170696871 | 0.256981421 | 0.62673744 |
| eqtl-a-ENSG00000168062 | | BATF2 | 0.023562475 | 0.195217606 | 0.906581497 |
| eqtl-a-ENSG00000170271 | | FAXDC2 | −0.023532901 | 0.009861354 | 0.018935798 |
| eqtl-a-ENSG00000204262 | | COL5A2 | −0.026899629 | 0.023579891 | 0.259185483 |
| eqtl-a-ENSG00000256660 | | CLEC12B | 0.069960975 | 0.016441175 | 6.28E−05 |

TABLE 4
Steiger Directionality Test Results for Candidate Genes and IPF in Mendelian Randomization Analysis

| | | | snp_r2.ex- | snp_r2.out- | correct_caus- | |
|---|---|---|---|---|---|---|
| id.exposure | outcome | SYMBOL | posure | come | al_direction | steiger_pval |
| eqtl-a-ENSG00000100024 | Idiopathic | UPB1 | 0.946726917 | 0.000392348 | TRUE | 0 |
| eqtl-a-ENSG00000104267 | pulmonary | CA2 | 1.351004555 | 0.000377716 | TRUE | NA |
| eqtl-a-ENSG00000134247 | fibrosis ∥ | PTGFRN | 0.102605529 | 0.000121573 | TRUE | 0 |
| eqtl-a-ENSG00000137265 | id: finn- | IRF4 | 0.047005254 | 0.000212465 | TRUE | 9.54E−239 |
| eqtl-a-ENSG00000144230 | b-IPF | GPR17 | 0.014971968 | 3.87E−05 | TRUE | 5.93E−66 |
| eqtl-a-ENSG00000148671 | | ADIRF | 0.066442541 | 7.27E−05 | TRUE | 0 |
| eqtl-a-ENSG00000161647 | | MPP3 | 0.004505628 | 2.64E−05 | TRUE | 4.81E−24 |
| eqtl-a-ENSG00000168062 | | BATF2 | 0.03769686 | 0.000225304 | TRUE | 1.20E−182 |
| eqtl-a-ENSG00000204262 | | COL5A2 | 0.207364895 | 0.000449171 | TRUE | 0 |

### 2.4 ADIRF, PTGFRN, and UPB1 Identified as Biomarkers for IPF

To identify robust feature genes, the eight causally associated key genes were subjected to machine learning-based selection. Least Absolute Shrinkage and Selection Operator (LASSO) regression analysis revealed that, when the penalty parameter λ was set to 0.0114, six genes—ADIRF, PTGFRN, UPB1, MPP3, BATF2, and IRF4—exhibited non-zero coefficients and were retained as candidate feature genes (FIGS. 4A-4B). In parallel, the Support Vector Machine-Recursive Feature Elimination (SVM-RFE) algorithm identified the optimal model with the highest accuracy, which included three feature genes: ADIRF, PTGFRN, and UPB1 (FIG. 4C).

A Venn diagram was employed to visualize the overlap between the LASSO- and SVM-RFE-derived feature sets, and the three intersecting genes-ADIRF, PTGFRN, and UPB1—were designated as candidate biomarkers for IPF (FIG. 4D).

Expression analysis in both the GSE150910 and GSE110147 datasets demonstrated that PTGFRN was significantly upregulated in IPF samples, whereas ADIRF and UPB1 were significantly downregulated, relative to normal controls (P<0.05) (FIGS. 4E-4F).

Receiver operating characteristic (ROC) curve analysis was conducted to assess the diagnostic performance of these biomarkers. In the GSE150910 and GSE110147 datasets, the area under the curve (AUC) values for ADIRF were 0.886 and 1.000, for PTGFRN were 0.925 and 1.000, and for UPB1 were 0.887 and 1.000, respectively—indicating high diagnostic accuracy (FIGS. 4G-4H).

### 2.5 MR Analysis of the Three Biomarkers

The causal relationship between the three identified biomarkers and IPF was further evaluated using Mendelian randomization analysis. The results showed that PTGFRN (OR=0.821; 95% CI: 0.719-0.937; P=0.003) and UPB1 (OR=0.954; 95% CI: 0.922-0.986; P=0.005) function as protective factors for IPF. Conversely, ADIRF (OR=1.299; 95% CI: 1.069-1.577; P=0.008) was identified as a risk factor.

For PTGFRN and UPB1, the inverse variance weighted (IVW) regression slopes in the scatter plots were negative, and the estimated MR effect sizes in the forest plots were less than zero, thereby supporting their protective roles in IPF (FIGS. 5A-5B). In contrast, ADIRF exhibited a positive IVW slope and effect size, reinforcing its pathogenic association (FIGS. 5A-5B).

Furthermore, funnel plots revealed a symmetric distribution of single nucleotide polymorphisms (SNPs) around the IVW estimate, indicating that the MR results adhered to the second law of Mendelian randomization-random distribution of instrumental variables (FIG. 5C).

### 2.6 Construction of a Predictive Nomogram for IPF Diagnosis

A diagnostic nomogram was constructed based on the expression levels of the three identified biomarkers using data from the GSE150910 dataset (FIG. 6A). The calibration curve demonstrated a slope approaching 1, indicating high predictive accuracy of the nomogram for IPF diagnosis (FIG. 6B). The receiver operating characteristic (ROC) curve analysis showed an area under the curve (AUC) of 0.932, reflecting excellent diagnostic specificity and sensitivity (FIG. 6C). Furthermore, decision curve analysis demonstrated that both the individual biomarkers and the nomogram provided notable clinical net benefit, with the nomogram outperforming the standalone biomarkers (FIG. 6D). These results underscore the diagnostic utility and predictive value of the nomogram constructed from the three biomarkers.

### 2.7 Artificial Neural Network (ANN) Diagnostic Model Exhibits High Accuracy

An ANN diagnostic model was developed using the expression profiles of the three biomarkers in the GSE150910 and GSE110147 datasets to distinguish IPF patients from non-IPF controls (FIGS. 7A-7B). The ROC curve analysis revealed AUC values of 0.942 and 1.000 for the two datasets, respectively (FIGS. 7C-7D), indicating that the ANN model possesses high diagnostic accuracy and robustness for IPF classification.

### 2.8 Biological Mechanism Elucidation of the Identified Biomarkers

Using the GeneMANIA database, a co-expression network comprising the three biomarkers and 20 interacting genes was constructed to investigate potential biological functions (FIG. 8A). Genes functionally associated with the biomarkers included DPYS, CPS1, CD9, and NIT1, among others. These genes were predominantly involved in biological processes such as “hydrolase activity acting on carbon-nitrogen bonds, in linear amides” and “skeletal muscle tissue regeneration.” Further, gene set enrichment analysis (GSEA) identified biological pathways associated with the three biomarkers. All three were enriched in pathways such as ribosome, oxidative phosphorylation, and olfactory transduction, among others (Supplementary Tables 3-5). Notably, ADIRF was involved in several immune-related pathways, including cell cycle, JAK-STAT signaling, Toll-like receptor signaling, and RIG-I-like receptor signaling. The top ten KEGG pathways based on enrichment scores are shown in FIGS. 8B-8D. SPEED2 upstream signaling analysis indicated that the three biomarkers were most significantly associated with the TGF-β and JAK-STAT signaling pathways (FIG. 8E).

2.9 Association of Biomarkers with Immune Cell Infiltration in IPF

As shown in FIG. 9A, immune infiltration analysis in the GSE150910 dataset revealed differential enrichment scores for 28 immune cell types between IPF and control samples. Wilcoxon test results identified 14 immune cell types with significant differences (P<0.05). Among these, five cell types—including activated B cells, CD56\hat{ }bright natural killer cells, immature B cells, immature dendritic cells, and memory B cells—were significantly enriched in IPF samples, whereas the remaining nine, including effector memory CD8+ T cells, γδ T cells, and monocytes, were reduced (FIG. 9B). Moreover, correlation analysis showed that the three biomarkers were significantly associated with most of the differentially expressed immune cells (|correlation coefficient|>0.3, P<0.05) (FIG. 9C). Notably, T helper 17 (Th17) cells exhibited the strongest positive correlations with ADIRF (cor=0.466, P<0.001) and UPB1 (cor=0.548, P<0.001), and the strongest negative correlation with PTGFRN (cor=0.548, P<0.001). In contrast, activated B cells showed an inverse correlation pattern compared to Th17 cells. Collectively, these findings suggest that differential immune cell infiltration may represent potential therapeutic targets for IPF.

### 2.10 Potential Regulatory Mechanisms and Drug Associations of Biomarkers

An mRNA-miRNA-lncRNA regulatory network was constructed, comprising two mRNAs (ADIRF and PTGFRN), nine miRNAs, and fourteen lncRNAs (FIG. 10A). Eight miRNAs were predicted to regulate PTGFRN (including hsa-miR-20b-5p, hsa-miR-17-5p, hsa-miR-139-5p, hsa-miR-129-5p, etc.), and one miRNA (hsa-miR-212-3p) was identified as a potential regulator of ADIRF. No miRNA was predicted to target UPB1. Moreover, NEAT1 and XIST were found to potentially regulate ADIRF through hsa-miR-212-3p, whereas MALAT1 could simultaneously regulate PTGFRN via hsa-miR-129-5p, hsa-miR-17-5p, hsa-miR-20b-5p, and hsa-miR-23b-3p.

Subsequently, 81 potential therapeutic compounds were identified using the CTD database. Among them, five compounds (Aflatoxin B1, Benzo (a) pyrene, Cyclosporine, Retinoic acid, and Valproic acid) showed associations with the three biomarkers (FIG. 10B). Disease association analysis via DisGeNET revealed that PTGFRN and ADIRF were both linked to prostate cancer, whereas UPB1 was associated with β-ureidopropionase deficiency, symptomatic infantile spasms, and other metabolic disorders (FIG. 10C).

### 2.11 Identification of 10 Cell Subpopulations in Single-Cell Transcriptomics

Following quality control, 20,242 qualified cells were retained for downstream single-cell RNA sequencing analysis. The top 2000 highly variable genes are visualized in FIG. 11A, and principal component analysis (PCA) identified 30 statistically significant PCs (P<0.05) (FIG. 11B). Unsupervised clustering and cell-type annotation resulted in 26 clusters corresponding to 10 major cell subpopulations: B cells, endothelial cells, epithelial cells, macrophages, monocytes, natural killer cells, CD34+ pro-B cells, smooth muscle cells, T cells, and tissue stem cells (FIGS. 11C-11D). A bubble plot illustrated the top five marker genes with highest expression levels in each subpopulation (FIG. 11E). Cell-cell communication analysis suggested the strongest interaction occurred between macrophages and epithelial cells (FIGS. 11F1-11F2), with the SCGB3A2-MARCO ligand-receptor pair representing the highest interaction index between macrophages and dendritic cells (FIG. 11G).

### 2.12 Tissue Stem Cells as Key Regulators in Single-Cell Profiles

Tissue stem cells exhibited high expression of the biomarkers, especially ADIRF, which showed significant differential expression between IPF and normal samples (FIGS. 12A-12B). Consequently, tissue stem cells were designated as key cellular components in IPF pathology. ReactomeGSA pathway enrichment analysis revealed that tissue stem cells were involved in IPF progression through several biological processes, including serotonin metabolism, β-Klotho-mediated ligand binding, and CYP2E1 reactions (FIG. 12C).

Following dimensionality reduction, tissue stem cells were classified into eight subclusters to further reconstruct their developmental trajectory (FIG. 12D). Pseudotime analysis revealed that clusters 0, 4, and 6 were at the initial stage, progressing through cluster 3 and subsequently branching toward clusters 5 and 2, eventually culminating in cluster 1 (FIGS. 12E1-12E3). Notably, the expression of ADIRF showed a declining trend along this trajectory (FIG. 12F).

### 2.13 Histological Changes in the Mouse Pulmonary Fibrosis Model: HE and Masson Staining

As shown in FIG. 13A, H&E staining revealed that lung tissues in the control group exhibited intact alveolar architecture with clear alveolar walls and septa, without signs of inflammation, congestion, or edema. In contrast, the model group displayed pronounced pathological alterations characterized by alveolitis, thickened alveolar septa, and infiltration by inflammatory cells including monocytes, neutrophils, plasma cells, and lymphocytes. Severe cases exhibited abscess formation, vascular inflammation, focal pulmonary edema, and macrophage exudation within alveolar cavities.

Masson's trichrome staining (FIG. 13B) demonstrated that control lungs had regular collagen fiber distribution in alveolar and vascular walls. However, the model group showed disrupted alveolar structure, thickened alveolar walls, and significantly increased collagen deposition (P<0.01), along with capillary dilation and disorganized extracellular matrix arrangement.

### 2.14 Protein Expression Validation of Biomarkers via Western Blotting

Western blot analysis (FIG. 14) showed that in comparison to the control group, PTGFRN protein expression was significantly upregulated (P<0.05) and UPB1 expression was significantly downregulated (P<0.05) in lung tissues of the pulmonary fibrosis model group.

### 3. Discussion

IPF is a chronic disease of unknown etiology with limited treatment options; aside from lung transplantation, there is currently no curative therapy available for IPF (PMID: 35486072). Emerging evidence suggests that AS may contribute to the pathogenesis of IPF (PMID: 24647608). In this study, we utilized publicly available gene expression data from IPF patients in the GEO database and conducted a comprehensive series of bioinformatics analyses-including differential expression analysis, weighted gene co-expression network analysis (WGCNA), functional enrichment analysis, Mendelian randomization, machine learning, expression and ROC validation, GeneMANIA, gene set enrichment analysis (GSEA), immune infiltration analysis, regulatory network analysis, drug prediction, and single-cell RNA sequencing analysis. Through this integrative approach, we identified three AS-related biomarkers associated with IPF: ADIRF, PTGFRN, and UPB1. These findings provide new insights into the regulatory role of AS in the progression of IPF.

ADIRF (Adipogenesis Regulatory Factor) is a novel regulator involved in fatty acid metabolism (PMID: 35196590). Recent studies have implicated ADIRF in the development of lung cancer. For instance, N6-methyladenosine (m6A)-modified ADIRF has been shown to suppress lung adenocarcinoma metastasis and serve as a potential prognostic biomarker (PMID: 37700507). Moreover, hypermethylation of the ADIRF promoter affects its expression and participates in the malignant transformation of bronchial epithelial cells induced by NNK, suggesting a positive regulatory role in NNK-induced lung carcinogenesis (PMID: 37777989). However, the role of ADIRF in the pathogenesis of IPF remains to be fully elucidated.

PTGFRN (Prostaglandin F2 receptor inhibitor) is a member of the tetraspanin superfamily, characterized by four transmembrane domains, and is implicated in various aspects of cell biology and physiology (PMID: 33503070). It has been reported that PTGFRN expression is upregulated in IPF tissues (PMID: 33760118), although its precise mechanistic role in disease development is still unclear.

UPB1 (Ureidopropionase, beta) is a transcription factor containing a basic helix-loop-helix (bHLH) domain, involved in regulating peroxidase gene expression and plays a critical role in maintaining the balance between cell proliferation and elongation (PMID: 38134185). Low expression levels of UPB1 have been correlated with shorter overall survival and lymph node metastasis in patients with lung adenocarcinoma, suggesting that UPB1 may serve as a prognostic marker in lung cancer (PMID: 37859333). However, the involvement of UPB1 in IPF has not been previously investigated, and further studies are warranted to explore and validate its potential role.

Pathway enrichment analysis revealed that the three identified biomarkers were mainly involved in pathways such as “Cysteine and methionine metabolism,” “Notch signaling pathway,” and “Ribosome.” Specifically, ADIRF was significantly enriched in immune-related pathways, including the “JAK-STAT signaling pathway” and “Toll-like receptor signaling pathway.”

Previous studies suggest that the JAK-STAT pathway may play a critical role in the pathogenesis of interstitial lung diseases (ILDs). This pathway can be activated by a variety of pro-fibrotic and pro-inflammatory cytokines (e.g., IL-6, IL-11, and IL-13), which are often upregulated in ILDs. The JAK-STAT pathway regulates key cellular processes essential to ILD progression (PMID: 34207510). In the Toll-like receptor (TLR) signaling pathway, TLRs are known to promote tissue repair or fibrosis in several diseases, including pulmonary fibrosis. However, their effects vary substantially depending on the cellular microenvironment (PMID: 27364757). Notably, Mogroside IIIE (MGIIIE), a novel antifibrotic compound, has been shown to attenuate bleomycin-induced pulmonary fibrosis in mice via the TLR4 pathway (PMID: 28280123).

Our immune infiltration analysis identified significant differences in the abundance of nine immune cell types (e.g., B cells, T cells, dendritic cells, and monocytes) between control and IPF groups. Among the biomarkers, ADIRF and UPB1 were negatively correlated with B cell abundance and positively correlated with T cells, whereas PTGFRN displayed the opposite trend. Existing studies have demonstrated the involvement of B and T cells in the pathogenesis of IPF. Activated B cells can mediate fibroblast activation and migration, contributing to inflammation and fibrotic progression in IPF, and antifibrotic agents have been shown to at least partially reverse these responses (PMID: 33689587). Conversely, IPF fibroblasts secrete high levels of pro-apoptotic factors, including pro-caspase 3, cytochrome c, HIF-1a, HTRA2-OMI, and TNER1, which may lead to T cell apoptosis upon brief exposure (PMID: 35222396). These findings suggest that ADIRF, UPB1, and PTGFRN may influence the progression of IPF through modulation of B and T cell populations.

Furthermore, tissue stem cells, which are capable of differentiating into multiple immune cell types—including B cells, T cells, and dendritic cells—may play a key role in IPF. Our analyses indicated that tissue stem cells were primarily enriched in pathways such as “Metabolism of serotonin,” “Beta-Klotho-mediated ligand binding,” and “CYP2E1 reactions.” While the relevance of these pathways to IPF remains unclear, they warrant further experimental investigation to elucidate their roles in disease progression.

Regulatory Network: In our study, we predicted a miRNA, hsa-mir-212-3p, to regulate both PTGFRN and ADIRF. Additionally, we discovered that lncRNAs regulate ADIRF via hsa-mir-212-3p, while MALAT1, a specific lncRNA, regulates PTGFRN through multiple miRNAs, including hsa-mir-129-5p, hsa-mir-17-5p, hsa-mir-20b-5p, and hsa-mir-23b-3p. Long non-coding RNAs (lncRNAs) have previously been reported to play a significant role in IPF. For example, in the fibrotic lung tissues of IPF patients, H19 Imprinted Maternally Expressed Transcript (H19) is upregulated and induces fibrosis via the TGFβ/SMAD3 signaling pathway, interacting with hsa-miR-140 (PMID: 32438606). Moreover, MALAT1 has been shown to regulate TGF-β1-induced endothelial-to-mesenchymal transition (EndMT), a process known to contribute to fibrosis in various diseases (PMID: 28535533). Based on these findings, we hypothesize that lncRNAs, by regulating miRNA (hsa-mir-212-3p), may influence the expression of PTGFRN and ADIRF, ultimately modulating signaling pathways that play a crucial role in the pathogenesis of IPF.

To verify the correlation between the identified genes and the progression of pulmonary fibrosis, we constructed a pulmonary fibrosis mouse model. Due to species-specific factors, ADIRF was found to be deleted in mice (PMID: 31945134), so our study focused on PTGFRN and UPB1 in the lung tissues of normal and model groups. Western blot (WB) results confirmed that PTGERN expression was significantly upregulated in the lung tissues of the model group, while the expression of UPB1 was reduced.

In conclusion, this study, based on bioinformatics approaches, is the first to identify biomarkers related to AS in IPF. We have identified three key genes (ADIRF, PTGFRN, and UPB1) associated with IPF and further analyzed their molecular mechanisms in IPF pathogenesis. We also validated the relationship between PTGFRN and UPB1 with pulmonary fibrosis progression through the mouse model. The discovery of ADIRF, PTGFRN, and UPB1 provides new directions for the treatment of IPF. However, further in-depth experiments are needed to elucidate the specific mechanisms involved.

What is claimed is:

1. A biomarker for idiopathic pulmonary fibrosis, wherein the biomarker comprises ADIRF, PTGFRN, and UPB1.

2. A use of the biomarker for IPF and/or a detection reagent thereof according to claim 1 in the preparation of a product for diagnosing the idiopathic pulmonary fibrosis.

3. A diagnostic kit for the idiopathic pulmonary fibrosis, wherein the diagnostic kit comprises a reagent for detecting expression levels of the IPF-related biomarkers according to claim 1.

4. A use of the biomarker for IPF and/or a detection reagent thereof according to claim 1 in screening of drugs or methods for prevention and/or treatment of the idiopathic pulmonary fibrosis.

5. The use according to claim 4, wherein the screening comprises detecting effects of candidate drugs or methods on expression levels of the biomarkers ADIRF, PTGFRN, or UPB1, and selecting the candidate drugs or methods capable of modulating the expression levels of ADIRF, PTGFRN, or UPB1 for further validation testing.

6. A use of the biomarker for the idiopathic pulmonary fibrosis and/or a modulator thereof according to claim 1 in the preparation of a medicament for treatment of the idiopathic pulmonary fibrosis.

7. The use according to claim 6, wherein the modulator comprises at least one selected from a group consisting of aflatoxin B1, benzo(a)pyrene, cyclosporine, retinoic acid, and valproic acid.

8. A pharmaceutical composition, wherein the pharmaceutical composition comprises a modulator of the IPF biomarker according to claim 1.

9. The pharmaceutical composition according to claim 8, wherein the pharmaceutical composition further comprises a pharmaceutically acceptable excipient.
